(19) United States
(12) Patent Application Publication
(10) Pub. No.: US 2026/0103473 A1
(43) Pub. Date: Apr. 16, 2026

(54) CITRATE SALT OF CYCLIN-DEPENDENT KINASE (CDK4/6) INHIBITOR, CRYSTAL FORM THEREOF, PREPARATION METHOD THEREFOR AND USE THEREOF

(71) Applicant: Avanc Pharmaceutical Co., Ltd., Jinzhou Liaoning (CN)

(72) Inventors: Kexin WANG, Jinzhou Liaoning (CN); Yuhui PENG, Jinzhou Liaoning (CN); Qiaoyou ZHU, Jinzhou Liaoning (CN); Wenshu HUANG, Jinzhou Liaoning (CN); Jiuzhi LIU, Jinzhou Liaoning (CN); Meixia JIN, Jinzhou Liaoning (CN); Jiangtian YU, Jinzhou Liaoning (CN); Shuang ZHU, Jinzhou Liaoning (CN)

(21) Appl. No.: 19/116,112

(22) PCT Filed: Sep. 13, 2023
(86) PCT No.: PCT/CN2023/118429

§ 371 Date: Mar. 27, 2025

(30) Foreign Application Priority Data
Sep. 28, 2022 (CN) 202211195360.2

(51) Int. Cl. C07D 495/04

(52) U.S. Cl. CPC C07D 495/04

(57) ABSTRACT

The present invention relates to a citrate salt of a compound of formula (I). The present invention also relates to a crystal form A, a crystal form B, a crystal form C, and a crystal form D of the citrate salt of the compound of formula (I). The present invention also relates to a pharmaceutical composition comprising at least one of the citrate salt of the compound of formula (I) of the present invention, and the crystal form A, the crystal form B, the crystal form C and the crystal form D of the citrate salt of the compound of formula (I) of the present invention. The present invention also relates to use of the citrate salt of the present invention, the crystal form A, the crystal form B, the crystal form C, and the crystal form D of the citrate salt of the present invention, and the pharmaceutical composition of the present invention in the preparation of a drug for treating, preventing or ameliorating abnormal cell proliferation. The present invention also relates to use of the citrate salt of the present invention, the crystal form A, the crystal form B, the crystal form C, and the crystal form D of the citrate salt of the present invention, and the pharmaceutical composition of the present invention, optionally in combination with a second therapeutic agent, in the preparation of a drug for treating, preventing or ameliorating abnormal cell proliferation.

This application claims the priority of Chinese Patent Application No. 202211195360.2, entitled “citrate salt of cyclin-dependent kinase inhibitor, crystal form thereof, preparation method therefor and use thereof”, filed on Sep. 28, 2022, the contents of which are incorporated herein by reference in their entirety.

### TECHNICAL FIELD

The present invention belongs to the field of medicinal chemistry and particularly relates to a citrate salt of a protein kinase inhibitor, a crystal form A, a crystal form B, a crystal form C and a crystal form D thereof, and preparation method therefor and use thereof.

### BACKGROUND

Hyperproliferative diseases such as cancer and inflammation have been attractive in academia for providing effective therapeutic means, and efforts have been made in this regard to identify and target specific mechanisms that play a role in proliferative diseases.

The development of tumors is closely related to cyclin-dependent kinases (CDKs) and gene mutations and regulatory abnormalities thereof, indicating that CDK inhibitors may be an effective anti-cancer therapy.

CDK is a serine/threonine protein kinase, which is a motive force for cell cycle and cell proliferation. CDK regulates the initiation, progression and completion of mammalian cell cycle and is critical to cell growth. Most known CDKs, including CDK1 through CDK9, are directly or indirectly involved in the cell cycle progression process. CDKs directly involved in cell cycle progression, such as CDK1-4 and CDK6, can be classified as G1, S or G2M phase enzymes. Abnormal proliferation is characteristic of cancer cells and CDK dysfunction occurs at high frequencies in many solid tumors.

CDKs and their associated proteins are critical in coordinating and driving the cell cycle in proliferating cells. Therefore, therapies targeting multiple CDKs or specific CDKs for the treatment of proliferative disorders, such as cancer, have great potential. CDK inhibitors can also be used to treat other diseases such as viral infections, autoimmune diseases, and neurodegenerative diseases. CDK targeted therapy may also be used in combination with other therapeutic drugs for the treatment of the above diseases.

Therefore, compounds with CDK inhibitory activity are of great significance for the prevention and treatment of cancer. Although CDK4/6 inhibitors have been reported in the literature, such as WO2010020675 and WO2012064805, many of them have short half lives or are toxic, and currently there are three types of CDK4/6 inhibitor antitumor drugs have been approved by the FDA in the world, which are palbociclib of Pfizer, ribociclib of Novartis, and abefcaciclib of Eli Lilly, respectively, which are used for the treatment of postmenopausal women in the advanced or metastatic breast cancer of HR+ and HER2−.

At present, the need for novel CDK4/6 inhibitors for treating hyperproliferative diseases is becoming more and more urgent, which has advantages in at least one of efficacy, stability, selectivity, safety, pharmacodynamics and pharmacokinetic characteristics. In addition, in view of the above considerations regarding the stability and safety of CDK4/6 inhibitors, there is an urgent need to seek salt forms and crystal forms thereof that can be used for long-acting treatment.

### SUMMARY

In one aspect, provided is a citrate salt of the compound of formula (I).

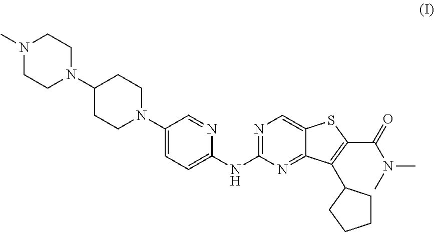

In an embodiment, the molar ratio of the compound of formula (I) to citric acid is about 1:1-1:3.

In another aspect, provided is a crystal form A of a citrate salt of the compound of formula (I), wherein the X-ray powder diffraction pattern of the crystal form A has characteristic diffraction peaks at the following 2θ angles: 17.9±0.2°, 18.4±0.2°, 19.2±0.2°, 19.5±0.2° and 20.5±0.2°.

In another aspect, provided is a crystal form B of a citrate salt of the compound of formula (I), wherein the X-ray powder diffraction pattern of the crystal form B has characteristic diffraction peaks at the following 2θ angles 8.3±0.2°, 11.2±0.2°, 14.1±0.2°, 16.7±0.2° and 18.4±0.2°.

In another aspect, provided is a crystal form C of a citrate salt of the compound of formula (I), wherein the X-ray powder diffraction pattern of the crystal form C has characteristic diffraction peaks at the following 2θ angles: 10.0±0.2°, 15.6±0.2°, 19.4±0.2°, 20.2±0.2° and 20.8±0.2°.

In another aspect, provided is a crystal form D of a citrate salt of the compound of formula (I), wherein the X-ray powder diffraction pattern of the crystal form D has characteristic diffraction peaks at the following 2θ angles: 14.0±0.2°, 16.8±0.2°, 18.6±0.2°, 19.8±0.2° and 24.6±0.2°.

In one aspect, provided is a process for preparing the crystal form A, the crystal form B, and the crystal form C of a citrate salt of the compound of formula (I).

In one aspect, provided is a pharmaceutical composition comprising one or more selected from the group consisting of (i) the citrate salt of the compound of formula (I) of the present invention; (ii) the crystal form A of the citrate salt of the compound of formula (I) of the present invention; (iii) the crystal form B of the citrate salt of the compound of formula (I) of the present invention; (iv) the crystal form C of the citrate salt of the compound of formula (I) of the present invention; (v) the crystal form D of the citrate salt of the compound of formula (I) of the present invention.

In one aspect, provided is use of the citrate salt, the crystal form A, the crystal form B, the crystal form C, the crystal form D of the compound of formula (I) of the present invention, and the composition of the present invention, in the manufacture of a medicament for treating, ameliorating, or preventing a condition responsive to inhibition of cyclin-dependent kinase 4/6.

In another aspect, provided is use of the citrate salt, the crystal form A, the crystal form B, the crystal form C, the crystal form D of the compound of formula (I) of the present invention, and the composition of the present invention, in the manufacture of a medicament for treating, ameliorating or preventing an abnormal cell proliferation.

In yet another aspect, provided is use of the citrate salt, the crystal form A, the crystal form B, the crystal form C, the crystal form D of the compound of formula (I) of the present invention, and the composition of the present invention, optionally in combination with a second therapeutic agent, in the manufacture of a medicament for treating, ameliorating, or preventing a condition responsive to inhibition of cyclin-dependent kinase 4/6.

In yet another aspect, provided is use of the citrate salt, the crystal form A, the crystal form B, the crystal form C, the crystal form D of the compound of formula (I) of the present invention, and the composition of the present invention, optionally in combination with a second therapeutic agent, in the manufacture of a medicament for treating, ameliorating or preventing an abnormal cell proliferation.

### BRIEF DESCRIPTION OF THE DRAWINGS

FIG. 1 is an X-ray powder diffraction (XRPD) pattern of the crystal form A of the citrate salt of the compound of formula (I).

FIG. 2 is a DSC spectrum of the crystal form A of the citrate salt of the compound of formula (I).

FIG. 3 is a TGA spectrum of the crystal form A of the citrate salt of the compound of formula (I).

FIG. 4 is an NMR spectrum of the crystal form A of the citrate salt of the compound of formula (I), wherein the test solvent is DMSO-d₆.

FIG. 5 is an NMR spectrum of the crystal form A of the citrate salt of the compound of formula (I), wherein the test solvent is D₂O.

FIG. 6 is an X-ray powder diffraction (XRPD) pattern of the crystal form B of the citrate salt of the compound of formula (I).

FIG. 7 is a DSC spectrum of the crystal form B of the citrate salt of the compound of formula (I).

FIG. 8 is a TGA spectrum of the crystal form B of the citrate salt of the compound of formula (I).

FIG. 9 is an NMR spectrum of the crystal form B of the citrate salt of the compound of formula (I), wherein the test solvent is DMSO-d₆.

FIG. 10 is an X-ray powder diffraction (XRPD) pattern of the crystal form C of the citrate salt of the compound of formula (I).

FIG. 11 is a DSC spectrum of the crystal form C of the citrate salt of the compound of formula (I).

FIG. 12 is a TGA spectrum of the crystal form C of the citrate salt of the compound of formula (I).

FIG. 13 is an NMR spectrum of the crystal form C of the citrate salt of the compound of formula (I), wherein the test solvent is DMSO-d₆.

FIG. 14 is an NMR spectrum of the crystal form C of the citrate salt of the compound of formula (I), wherein the test solvent is D₂O.

FIG. 15 is an X-ray powder diffraction (XRPD) pattern of the crystal form D of the citrate salt of the compound of formula (I).

FIG. 16 is a DSC spectrum of the crystal form D of the citrate salt of the compound of formula (I).

FIG. 17 is a TGA spectrum of the crystal form D of the citrate salt of the compound of formula (I).

FIG. 18 is a single crystal diagram of the crystal form D of the citrate salt of the compound of formula (I).

FIG. 19 is an X-ray powder diffraction (XRPD) pattern of the crystal form E of the citrate salt of the compound of formula (I).

FIG. 20 is an X-ray powder diffraction (XRPD) pattern of the crystal form F of the citrate salt of the compound of formula (I).

FIG. 21 is an X-ray powder diffraction (XRPD) pattern of the crystal form G of the citrate salt of the compound of formula (I).

FIG. 22 is an X-ray powder diffraction (XRPD) pattern of the crystal form G of the citrate salt of the compound of formula (I) after being stored at room temperature for 1 day.

FIG. 23 is a DVS curve diagram of the crystal form A of the citrate salt of the compound of formula (I).

FIG. 24 is an XRPD pattern of the crystal form A of the citrate salt of the compound of formula (I) before and after DVS test.

FIG. 25 is a DVS curve diagram of the crystal form B of the citrate salt of the compound of formula (I).

FIG. 26 is an XRPD pattern of the crystal form B of the citrate salt of the compound of formula (I) before and after DVS test.

FIG. 27 is a DVS curve diagram of the crystal form C of the citrate salt of the compound of formula (I).

FIG. 28 is an XRPD pattern of the crystal form C of the citrate salt of the compound of formula (I) before and after DVS test.

FIG. 29 is a DVS curve diagram of the crystal form D of the citrate salt of the compound of formula (I).

FIG. 30 is an XRPD pattern of the crystal form D of the citrate salt of the compound of formula (I) before and after DVS test.

FIG. 31 is a stability test XRPD pattern of the crystal form A of the citrate salt of the compound of formula (I).

FIG. 32 is a stability test XRPD pattern of the crystal form B of the citrate salt of the compound of formula (I).

FIG. 33 is a stability test XRPD pattern of the crystal form C of the citrate salt of the compound of formula (I).

FIG. 34 is a stability test XRPD pattern of the crystal form D of the citrate salt of the compound of formula (I).

### DETAILED DESCRIPTION

The present invention will be further described in detail below. Such description is for illustrative purposes and is not meant to be a limitation of the present invention. Other advantages and effects of the present invention will be readily apparent to those skilled in the art from this disclosure. The present invention may also be implemented or applied in other different embodiments. Those skilled in the art can make various modifications and variations without departing from the spirit of the present invention.

### General Definitions and Terminology

All publications, patent applications, patents, and other references mentioned herein are incorporated by reference in their entirety unless otherwise indicated.

Unless defined otherwise, all technical and scientific terms used herein have the same meaning as commonly understood by one of ordinary skill in the art to which the present invention belongs. In case of conflict, the definitions provided herein control.

Unless otherwise specified, all percentages, parts, ratios, etc. are by weight.

When quantities, concentrations, or other values or parameters are given as ranges, preferred ranges, or preferred upper and lower values or particular values, it is to be understood that all ranges formed from paired values of any upper range or preferred value and any lower range or preferred value are specifically disclosed, regardless of whether ranges are separately in disclosed. Where a range of numerical values is recited herein, unless otherwise stated, the range is intended to include the endpoints thereof, and all integers and fractions within the range. The scope of the present invention is not limited to the specific values recited when defining a range. For example, “1-20” encompasses 1, 2, 3, 4, 5, 6, 7, 8, 9, 10, 11, 12, 13, 14, 15, 16, 17, 18, 19, 20, and any sub-range consisting of any two of them, e.g., 2-6, 3-5, 2-10, 3-15, 4-20, 5-19, etc. For example, “1:1-1:5” encompasses 1:1, 1:2, 1:3, 1:4, 1:5, and any sub-range subsumed by any two of these values, e.g., 1:1-1:4, 1:1-1:3, 1:1-1:2, 1:2-1:4, 1:2-1:3, etc.

As used herein, the terms “about,” “approximately,” when used in conjunction with a numerical variable generally mean that the value of the variable and all values of the variable are within experimental error (e.g., within a 95% confidence interval for the mean) or within ±10% of the specified value, or a broader range.

As used herein, the term “selected from” refers to one or more elements of a group listed thereafter, independently selected, and may include a combination of two or more elements.

As used herein, the term “one or more” or “at least one” refers to one, two, three, four, five, six, seven, eight, nine, or more.

Unless otherwise indicated, the terms “a combination thereof” and “mixtures thereof” mean a multi-component mixture of the elements, e.g., two, three, four, and up to a maximum possible multi-component mixture.

In addition, the number of parts or components of the present invention is not previously indicated, indicating that there is no limit to the number of the occurrences (or present) of parts or components. Therefore, it should be read as including one or at least one, and the singular form of a part or component also includes the plural, unless the value clearly indicates the singular.

As used herein, the term “optional” or “optionally” means that the subsequently described event or circumstance may or may not occur, and that the description includes instances where said event or circumstance occurs and instances where said event or circumstance does not occur.

As used herein, the terms “comprising,” “including,” “having,” “containing,” or “involving,” and variations thereof herein, are intended to be inclusive or open-ended and do not exclude additional, unrecited elements or method steps. Those skilled in the art should understand that the above terms such as “comprising” encompass the meaning of “consisting of.” The expression “consisting of” excludes any element, step, or ingredient not specified. The expression “consisting essentially of” refers to a range limited to a specified element, step, or ingredient plus an optional element, step, or ingredient that does not materially affect the basic and novel characteristic(s) of the claimed subject matter. It should be understood that the expression “comprising” encompasses the expressions “consisting essentially of” and “consisting of.”

As used herein, the term “pharmaceutically acceptable” refers to contact with the tissue of a patient within a normal medical judgment range without undue toxicity, irritation, allergic response, etc., has a reasonable benefit/disadvantage ratio and is effective for the intended purpose.

As used herein, the term “crystal form” or “crystal” refers to any solid substance that exhibits a three-dimensional ordering, as opposed to an amorphous substance, which produces a characteristic X-ray powder diffraction pattern with well-defined peaks.

As used herein, the term “amorphous” refers to any solid substance that is three-dimensionally unordered.

As used herein, the term “hydrate” refers to salts of compounds provided herein, further comprising stoichiometric or non-stoichiometric amounts of water bound by non-covalent intermolecular forces.

As used herein, the term “mixture” refers to a substance mixed from two or more substances. In the present invention, a “mixture” is connected or combined in a non-covalent bond, and has a molecular formula, composition ratio (molar ratio or mass ratio) of a specific value or a specific numerical range, and has stable physicochemical properties and biological properties.

As used herein, the terms “citrate salt” and “citrate” may be used interchangeably and refer to a salt form formed by a free base compound and citric acid.

As used herein, the term “citrate salt of the compound of formula (I)” refers to the salt formed by the free base of the compound of formula (I) and citric acid. The term “monocitrate salt of the compound of formula (I)” means that the molar ratio of the compound of formula (I) to citric acid in the citrate salt is about 1:1. The term “dicitrate salt of the compound of formula (I)” means that the molar ratio of the compound of formula (I) to citric acid in the citrate salt is about 1:2. The term “crystal form of citrate salt of the compound of formula (I)” refers to a citrate salt of the compound of formula (I) in crystal form. Depending on whether the crystal form contains a solvent molecule, it may be a crystal form of a solvate, or the crystal form may not contain a solvent molecule. The solvent may be methanol or water. In the case where the solvent is water, it may also be referred to as a corresponding hydrate.

As used herein, the term “pharmaceutical composition” refers to an active ingredient, optionally in combination with one or more pharmaceutically acceptable chemical components, such as, but not limited to, carriers and/or excipients. The active ingredient may be, for example, a citrate salt, a crystal form or a combination thereof of the compound of formula (I).

As used herein, the term “active ingredient” refers to a chemical entity that can effectively treat or prevent a target disease or condition.

As used herein, the term “pharmaceutically acceptable carrier” refers to those carriers that do not significantly irritate the organism and do not impair the biological activity and properties of the active compound, including, but not limited to, any glidant, sweetener, diluent, preservative, dye/colorant, flavor enhancer, surfactant, wetting agent, dispersing agent, disintegrating agent, suspending agent, stabilizer, isotonic agent, solvent or emulsifier acceptable for use in humans or animals (e.g., livestock). Non-limiting examples of such carriers include calcium carbonate, calcium phosphate, various sugars and various starches, cellulose derivatives, gelatin, vegetable oils and polyethylene glycols and the like. Additional information regarding carriers can be found in Remington: The Science and Practice of Pharmacy, 21st Ed., Lippincott, Williams & Wilkins (2005), the contents of which are incorporated herein by reference.

As used herein, the terms “administration” or “administering” and the like refer to a method by which a compound or composition can be delivered to a desired site of biological action. These methods include, but are not limited to, oral, parenteral (including intravenous, subcutaneous, intraperitoneal, intramuscular, intravascular injection or infusion), topical, rectal administration, and the like.

For a drug or pharmacologically active agent, the term “effective amount” refers to a sufficient amount of a drug or agent that is non-toxic but achieves the desired effect. For oral dosage forms of the present invention, an “effective amount” of one active substance in a composition may be the amount required to achieve the desired effect when combined with another active substance in the composition. The determination of the effective amount varies from person to person, depending on the age and general condition of the receptor, and also depending on the specific active substance, and the appropriate effective amount in each case can be determined by those skilled in the art according to routine experimentation.

As used herein, the term “non-covalent bond form” refers to weak interactions between molecules other than covalent bonds, including but not limited to hydrogen bond, van der Waals force, salt bond, hydrophobic force, aromatic ring stacking interaction, x-x stacking, halogen bond, etc.

As used herein, the term “X-ray powder diffraction pattern (XRPD pattern)” refers to an experimentally observed diffraction pattern or a parameter derived therefrom. XRPD patterns are generally characterized by peak position (abscissa) and/or peak intensity (ordinate).

In an X-ray powder diffraction (XRPD or XRD) spectrum, the diffraction pattern obtained from a crystalline compound tends to be characteristic for a particular crystal form, where the relative intensity of the band (especially at low angles) may vary due to the dominant orientation effects produced by differences in crystallization conditions, particle size, and other determination conditions. Therefore, the relative intensities of the diffraction peaks are not characteristic of the targeted crystal form. When determining whether it is the same as a known crystal form, more attention should be paid to the relative positions of the peaks rather than their relative intensities. In addition, there may be slight errors in the position of the peak for any given crystal form, which is also well known in the crystallographic arts. For example, the position of the peak may be shifted due to changes in temperature as the sample is analyzed, sample movement or calibration of the instrument, etc., and the measurement error of the 20 value is sometimes about ±0.2°. Therefore, this error should be taken into account when determining each crystal form structure. If the crystalline forms of the present invention are described as substantially as shown in the specified drawings, the term “substantially” is also intended to encompass such differences in the diffraction peak positions.

In the XRPD pattern, a 2θ angle or a crystal plane distance d is usually used to represent a peak position, and there is a simple conversion relationship between them: d=N/2 sin θ, where d represents the crystal plane distance, λ represents the wavelength of incident X-ray, and θ is the diffraction angle. For the same crystal form of the same compound, the peak positions of the XRPD pattern have similarity as a whole, and the relative intensity error may be relatively large. It should also be noted that in the identification of the mixture, some diffraction lines may be missing due to factors such as content decrease, and at this time, it is not necessary to rely on all the spectral bands observed in the high-purity sample, and even one spectral band may be characteristic for a given crystal.

As used herein, the term “2θ” refers to the peak position in degrees based on the experimental setup of the X-ray diffraction experiment, and is generally the abscissa unit in the diffraction pattern. If the reflection is diffracted when the incident beam forms a θ angle with a certain lattice plane, the experimental setup requires recording the reflected beam at 2θ angle. It should be understood that the specific 2θ values for the particular crystal form mentioned herein are intended to represent the 2θ values measured in degrees using the X-ray diffraction experimental conditions described herein.

As used herein, the term “thermogravimetric analysis (TGA) spectrum” refers to a curve recorded by a thermogravimetric analyzer.

As used herein, the term “differential scanning calorimetry (DSC) spectrum” refers to a curve recorded by a differential scanning calorimeter.

As used herein, the term “nuclear magnetic resonance (¹H-NMR) spectrum” refers to a signal peak recorded by a nuclear magnetic resonance instrument.

### Salt of the Compound of Formula (I)

Provided is, a citrate salt of a protein kinase inhibitor, i.e. the compound of formula (I).

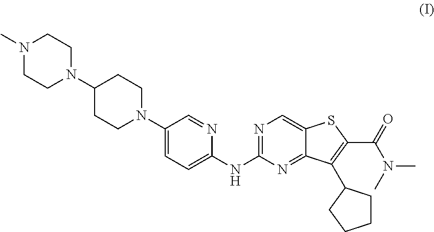

In an embodiment, the molar ratio of the compound of formula (I) to citric acid is about 1:1-1:3. In a preferred embodiment, in the citrate salt of the compound of formula (I), the molar ratio of the compound to citric acid is about 1:1-1:2. In a more preferred embodiment, in the citrate salt of the compound of formula (I), the molar ratio of the compound to citric acid is about 1:2. For example, about 1:1, about 1:2, about 1:3.

In a preferred embodiment, in the citrate salt of the compound of formula (I), the molar ratio of the compound to citric acid is about 1:2, which has the following chemical structure:

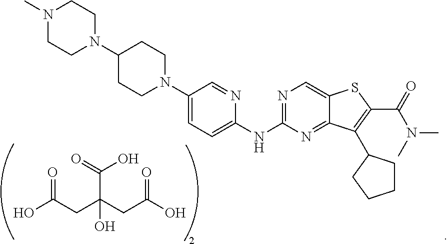

In another preferred embodiment, in the citrate salt of the compound of formula (I), the molar ratio of the compound to citric acid is about 1:1, which has the following chemical structure:

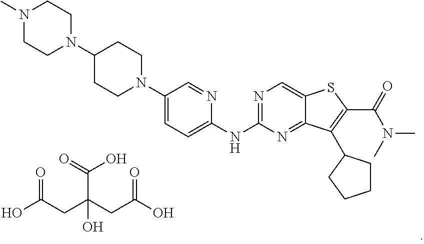

In the citrate salts of the compound of formula (I) of the present invention, an appropriate molar ratio of the compound to citric acid is beneficial to make the citrate salt have excellent properties. An excessively high molar ratio of the compound to citric acid is not conducive to obtaining the citrate salt with excellent properties. For example, an excessively high molar ratio of the compound to citric acid reduces the stability and solubility of the citrate salt. An unduly low molar ratio of the compound to citric acid is not conducive to obtaining the citrate salt with excellent properties. For example, an unduly low molar ratio of the compound to citric acid reduces the stability and solubility of the citrate salt.

### Crystal Forms of Citrate Salt of the Compound of Formula (I)

### Crystal Form A

In one aspect, provided is a crystal form A of a citrate salt of the compound of formula (I).

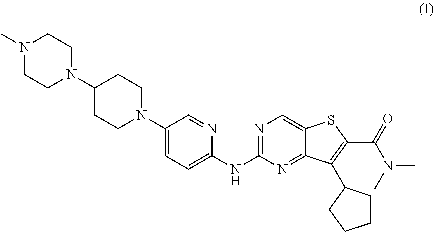

In an embodiment, in the crystal form A of the citrate salt of the compound of formula (I) of the present invention, the molar ratio of the compound to citric acid is about 1:2.

In an embodiment, the crystal form A of the citrate salt of the compound of formula (I) of the present invention does not contain crystallization water.

In an embodiment, the X-ray powder diffraction pattern of the crystal form A of the citrate salt of the compound of formula (I) of the present invention has characteristic diffraction peaks at the following 2θ angles: 17.9±0.2°, 18.4±0.2°, 19.2±0.2°, 19.5±0.2° and 20.5±0.2°.

In a preferred embodiment, the X-ray powder diffraction pattern of the crystal form A of the citrate salt of the compound of formula (I) of the present invention has characteristic diffraction peaks at the following 2θ angles: 9.8±0.2°, 11.6±0.2°, 15.0±0.2°, 17.2±0.2°, 17.6±0.2°, 17.9±0.2°, 18.4±0.2°, 19.2±0.2°, 19.5±0.2° and 20.5±0.2°.

In a more preferred embodiment, the X-ray powder diffraction pattern of the crystal form A of the citrate salt of the compound of formula (I) of the present invention has characteristic diffraction peaks at the following 2θ angles: 3.8±0.2°, 9.8±0.2°, 11.2±0.2°, 11.6±0.2°, 11.8±0.2°, 12.6±0.2°, 12.8±0.2°, 15.0±0.2°, 16.8±0.2°, 17.2±0.2°, 17.6±0.2°, 17.9±0.2°, 18.4±0.2°, 19.2±0.2°, 19.5±0.2°, 20.5±0.2°, 21.1±0.2°, 21.9±0.2°, 24.7±0.2°, and 25.2±0.2°.

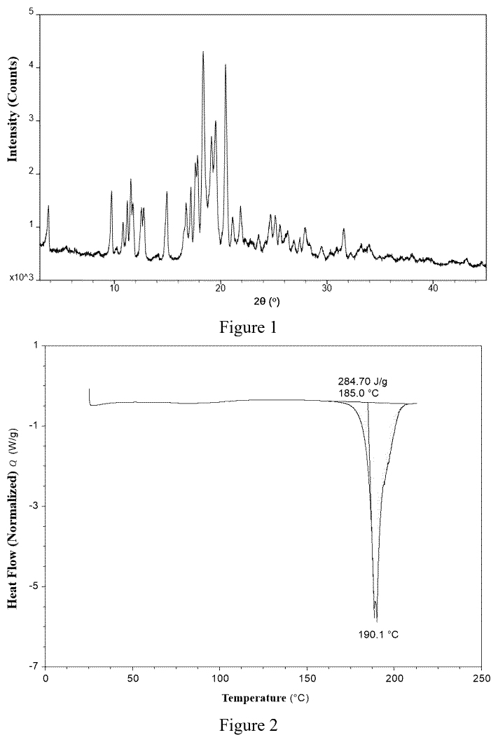

In a particularly preferred embodiment, the XRPD pattern of the crystal form A is as shown in FIG. 1.

In a specific embodiment, the XRPD pattern analysis data of the crystal form A is as shown in Table 1.

TABLE 1
XRPD pattern analysis data of crystal form A

| | 2θ | Interplanar | Relative |
|---|---|---|---|
| Number | angle(°) | distance (Å) | intensity(%) |
| 1 | 3.8 | 23.24012 | 31.5 |
| 2 | 9.8 | 9.09810 | 39.1 |
| 3 | 10.8 | 8.22219 | 24.9 |
| 4 | 11.2 | 7.91799 | 33.8 |
| 5 | 11.6 | 7.68723 | 44.4 |
| 6 | 11.8 | 7.55191 | 33.8 |
| 7 | 12.6 | 7.09239 | 30.9 |
| 8 | 12.8 | 6.95634 | 31.9 |
| 9 | 15.0 | 5.97243 | 38.9 |
| 10 | 16.8 | 5.33833 | 32.8 |
| 11 | 17.2 | 5.21167 | 38.6 |
| 12 | 17.6 | 5.09483 | 46.5 |
| 13 | 17.9 | 5.02232 | 52.7 |
| 14 | 18.4 | 4.88700 | 100.0 |
| 15 | 19.2 | 4.69628 | 62.9 |
| 16 | 19.5 | 4.60522 | 69.8 |
| 17 | 20.5 | 4.40666 | 93.4 |
| 18 | 21.1 | 4.27819 | 27.4 |
| 19 | 21.9 | 4.13377 | 32.6 |
| 20 | 23.6 | 3.84994 | 19.3 |
| 21 | 24.7 | 3.68437 | 28.7 |
| 22 | 25.2 | 3.62315 | 27.4 |
| 23 | 25.6 | 3.56756 | 23.7 |
| 24 | 26.2 | 3.49578 | 20.4 |
| 25 | 26.4 | 3.46989 | 20.3 |
| 26 | 26.9 | 3.40855 | 17.6 |
| 27 | 27.4 | 3.34368 | 18.0 |
| 28 | 28.0 | 3.28580 | 23.1 |
| 29 | 28.4 | 3.23974 | 15.7 |
| 30 | 29.5 | 3.12763 | 14.9 |
| 31 | 31.6 | 2.94034 | 22.8 |
| 32 | 33.3 | 2.80866 | 15.3 |
| 33 | 34.0 | 2.75593 | 15.3 |
| 34 | 43.1 | 2.25308 | 9.7 |

In an embodiment, the differential scanning calorimetry curve (DSC) of the crystal form A of the citrate salt of the compound of formula (I) of the present invention has an endothermic peak at 190.1±3° C.

In an embodiment, the DSC spectrum of the crystal form A of the citrate salt of the compound of formula (I) of the present invention is as shown in FIG. 2.

In an embodiment, the thermogravimetric analysis curve (TGA) of the crystal form A of the citrate salt of the compound of formula (I) of the present invention has a weight loss of 1.2% at 120.52±3° C. In an embodiment, the thermogravimetric analysis curve (TGA) of the crystal form A of the citrate salt of the compound of formula (I) of the present invention has a weight loss of 38.4% at 140−300=3° C.

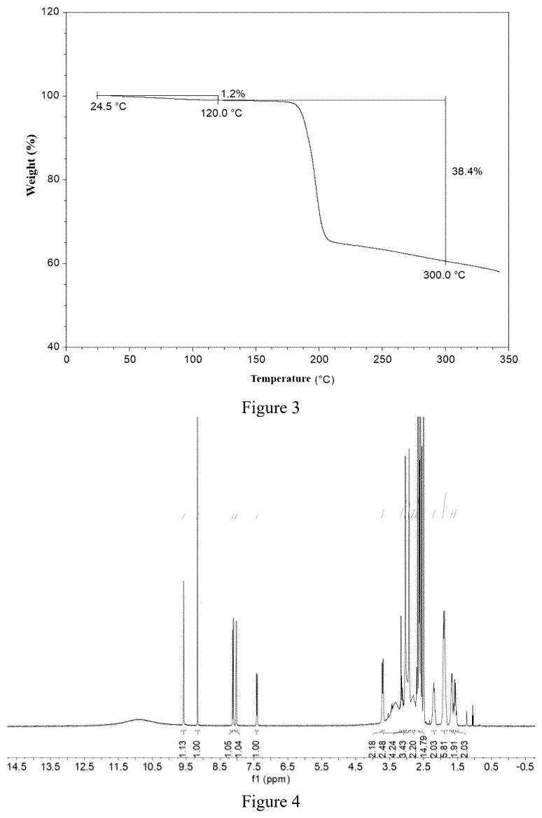

In an embodiment, the TGA spectrum of the crystal form A of the citrate salt of the compound of formula (I) of the present invention is as shown in FIG. 3.

### Crystal Form B

In one aspect, provided is a crystal form B of a citrate salt of the compound of formula (I).

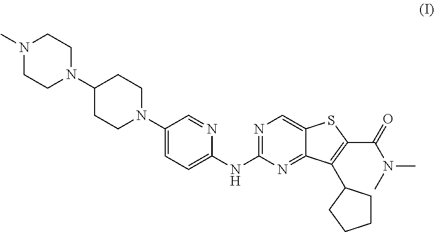

In an embodiment, in the crystal form B of the citrate salt of the compound of formula (I) of the present invention, the molar ratio of the compound to citric acid is about 1:2.

In another embodiment, in the crystal form B of the citrate salt of the compound of formula (I) of the present invention, the crystal form B is a crystal form of hydrate of the citrate salt.

In a specific embodiment, in the crystal form B of the citrate salt of the compound of formula (I) of the present invention, the molar ratio of the citrate salt of the compound of formula (I) to crystallization water is about 1:2.

In an embodiment, the X-ray powder diffraction pattern of the crystal form B of the citrate salt of the compound of formula (I) of the present invention has characteristic diffraction peaks at the following 2θ angles: 8.3±0.2°, 11.2±0.2°, 14.1±0.2°, 16.7±0.2° and 18.4=0.2°.

In a preferred embodiment, the X-ray powder diffraction pattern of the crystal form B of the citrate salt of the compound of formula (I) of the present invention has characteristic diffraction peaks at the following 2θ angles: 5.3±0.2°, 7.0±0.2°, 8.3±0.2°, 11.2±0.2°, 11.8±0.2°, 14.1±0.2°, 14.8±0.2°, 16.0±0.2°, 16.7±0.2°, 18.4±0.2°, 22.5±0.2°, 22.8±0.2°, 23.9±0.2°, 24.4±0.2° and 25.0±0.2°.

In a more preferred embodiment, the X-ray powder diffraction pattern of the crystal form B of the citrate salt of the compound of formula (I) of the present invention has characteristic diffraction peaks at the following 2θ angles: 5.3±0.2°, 7.0±0.2°, 8.3±0.2°, 10.2±0.2°, 11.2±0.2°, 11.8±0.2°, 13.2±0.2°, 14.1±0.2°, 14.8±0.2°, 16.0=0.2°, 16.7±0.2°, 18.4±0.2°, 19.7±0.2°, 20.1±0.2°, 21.0±0.2°, 21.3±0.2°, 22.5±0.2°, 22.8±0.2°, 23.0±0.2°, 23.9±0.2°, 24.4=0.2°, and 25.0±0.2°.

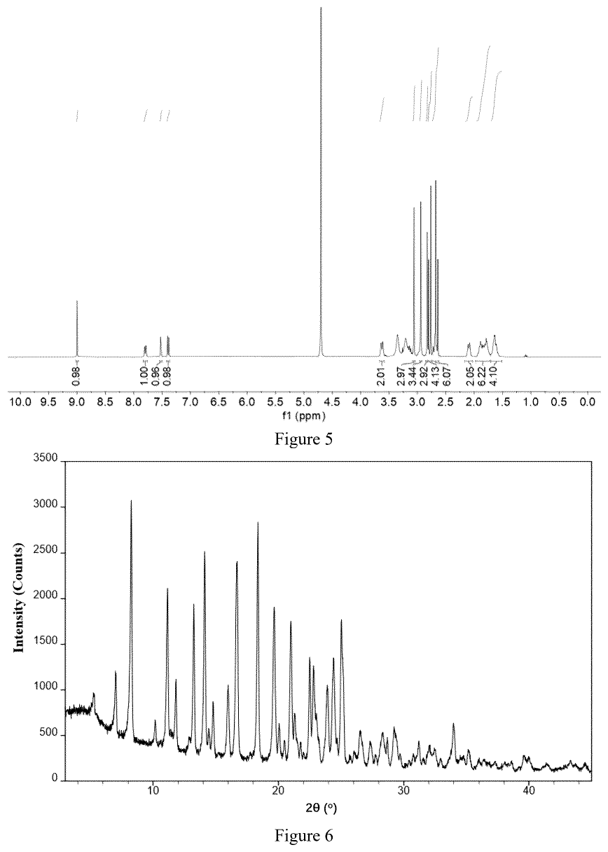

In an embodiment, the XRPD pattern of the crystal form B is as shown in FIG. 6.

In a specific embodiment, the XRPD pattern analysis data of the crystal form B is as shown in Table 2.

TABLE 2
XRPD pattern analysis data of crystal form B

| | 2θ | Interplanar | Relative |
|---|---|---|---|
| Number | angle(°) | distance (Å) | intensity(%) |
| 1 | 5.3 | 16.72669 | 31.2 |
| 2 | 7.0 | 12.60936 | 37.5 |
| 3 | 8.3 | 10.71677 | 100.0 |
| 4 | 10.2 | 8.72591 | 21.9 |
| 5 | 11.2 | 7.96400 | 68.7 |
| 6 | 11.4 | 7.78316 | 18.1 |
| 7 | 11.8 | 7.52708 | 35.4 |
| 8 | 12.9 | 6.91449 | 15.1 |
| 9 | 13.2 | 6.71925 | 63.1 |
| 10 | 14.1 | 6.31454 | 81.8 |
| 11 | 14.4 | 6.17404 | 18.9 |
| 12 | 14.8 | 6.03461 | 28.4 |
| 13 | 16.0 | 5.58957 | 34.3 |
| 14 | 16.7 | 5.35870 | 77.6 |
| 15 | 17.8 | 5.04745 | 10.5 |
| 16 | 18.4 | 4.88700 | 92.2 |
| 17 | 19.7 | 4.57569 | 62.0 |
| 18 | 20.1 | 4.48948 | 20.6 |
| 19 | 20.5 | 4.40395 | 14.6 |
| 20 | 21.0 | 4.29861 | 57.0 |
| 21 | 21.3 | 4.24045 | 24.2 |
| 22 | 21.8 | 4.15512 | 14.0 |
| 23 | 22.0 | 4.10800 | 10.2 |
| 24 | 22.5 | 4.02604 | 44.2 |
| 25 | 22.8 | 3.97333 | 41.1 |
| 26 | 23.0 | 3.93478 | 23.5 |
| 27 | 23.9 | 3.80014 | 34.3 |
| 28 | 24.4 | 3.72710 | 44.0 |
| 29 | 24.7 | 3.68804 | 15.4 |
| 30 | 25.0 | 3.64271 | 56.5 |
| 31 | 25.7 | 3.55057 | 8.7 |
| 32 | 26.0 | 3.51054 | 10.6 |
| 33 | 26.5 | 3.45392 | 17.6 |
| 34 | 27.3 | 3.35852 | 14.3 |
| 35 | 27.8 | 3.30580 | 9.3 |
| 36 | 28.3 | 3.25077 | 17.2 |
| 37 | 28.7 | 3.20850 | 15.7 |
| 38 | 29.2 | 3.15449 | 19.5 |
| 39 | 29.7 | 3.10628 | 9.7 |
| 40 | 30.4 | 3.04063 | 7.3 |
| 41 | 30.8 | 3.01133 | 9.9 |
| 42 | 31.2 | 2.97252 | 14.5 |
| 43 | 31.6 | 2.94253 | 8.4 |
| 44 | 32.1 | 2.90259 | 12.9 |
| 45 | 32.5 | 2.86804 | 11.2 |
| 46 | 33.0 | 2.83245 | 8.3 |
| 47 | 34.0 | 2.75593 | 20.3 |
| 48 | 34.5 | 2.71908 | 9.4 |
| 49 | 34.8 | 2.70289 | 8.9 |
| 50 | 35.1 | 2.67642 | 11.5 |
| 51 | 36.0 | 2.62290 | 7.8 |
| 52 | 36.3 | 2.59997 | 7.3 |
| 53 | 37.3 | 2.54251 | 7.0 |
| 54 | 38.1 | 2.49912 | 6.7 |
| 55 | 38.6 | 2.46888 | 6.8 |
| 56 | 39.6 | 2.41721 | 8.6 |
| 57 | 40.0 | 2.39669 | 8.4 |
| 58 | 41.4 | 2.32993 | 6.2 |
| 59 | 43.3 | 2.24595 | 7.5 |
| 60 | 44.5 | 2.19935 | 6.8 |

In an embodiment, the differential scanning calorimetry curve (DSC) of the crystal form B of the citrate salt of the compound of formula (I) of the present invention has an endothermic peak at 81.0±3° C., 133.2±3° C., 150.6±3° C. and 177.9±3° C., respectively.

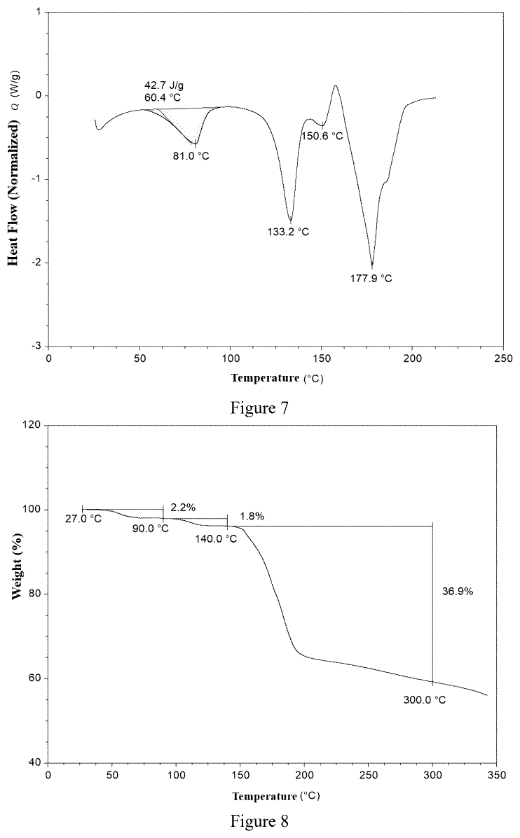

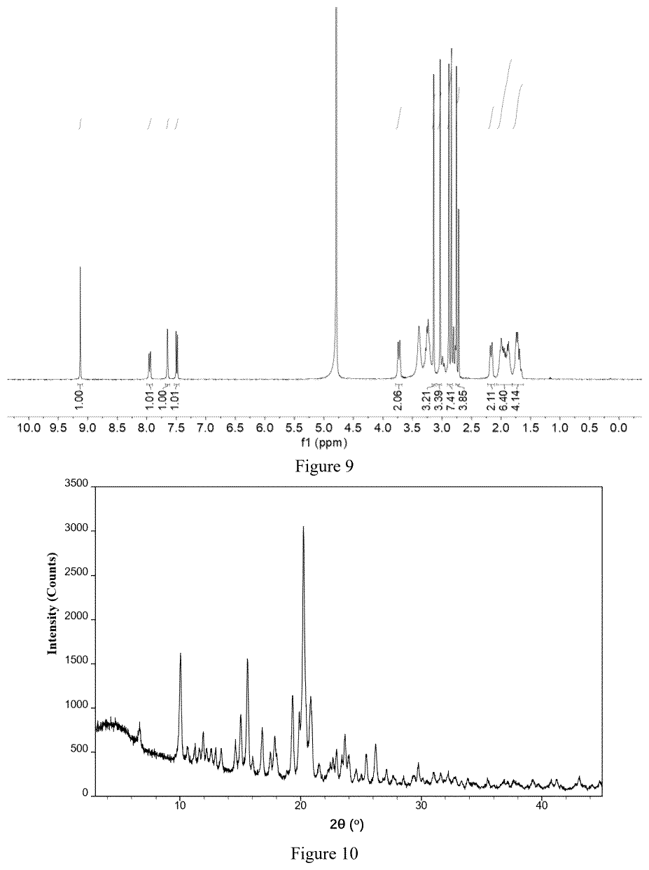

In an embodiment, the DSC spectrum of the crystal form B of the citrate salt of the compound of formula (I) of the present invention is as shown in FIG. 7.

In an embodiment, the thermogravimetric analysis curve (TGA) of the crystal form B of the citrate salt of the compound of formula (I) of the present invention has a weight loss of 2.2% at 90±3° C. In an embodiment, the thermogravimetric analysis curve (TGA) of the crystal form B of the citrate salt of the compound of formula (I) of the present invention has a weight loss of 1.8% at 90-140±3° C. In an embodiment, the thermogravimetric analysis curve (TGA) of the crystal form B of the citrate salt of the compound of formula (I) of the present invention has a weight loss of 36.9% at 140-300±3º° C.

In an embodiment, the TGA spectrum of the crystal form B of the citrate salt of the compound of formula (I) of the present invention is as shown in FIG. 8.

Crystal form C In another aspect, provided is a crystal form C of a citrate salt of the compound of formula (I).

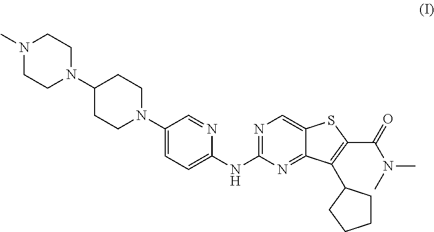

In an embodiment, in the crystal form C of the citrate salt of the compound of formula (I) of the present invention, the molar ratio of the compound to citric acid is about 1:1.

In an embodiment, the crystal form C of the citrate salt of the compound of formula (I) of the present invention does not contain crystallization water.

In an embodiment, the X-ray powder diffraction pattern of the crystal form C of the citrate salt of the compound of formula (I) of the present invention has characteristic diffraction peaks at the following 2θ angles: 10.0±0.2°, 15.6±0.2°, 19.4±0.2°, 20.2±0.2° and 20.8±0.2°.

In a preferred embodiment, the X-ray powder diffraction pattern of the crystal form C of the citrate salt of the compound of formula (I) of the present invention has characteristic diffraction peaks at the following 2θ angles: 6.7±0.2°, 10.0±0.2°, 11.9±0.2°, 15.0±0.2°, 15.6±0.2°, 16.8±0.2°, 19.4±0.2°, 19.9±0.2°, 20.2±0.2° and 20.8±0.2°.

In a more preferred embodiment, the X-ray powder diffraction pattern of the crystal form C of the citrate salt of the compound of formula (I) of the present invention has characteristic diffraction peaks at the following 2θ angles: 6.7±0.2°, 10.0±0.2°, 10.6±0.2°, 11.2±0.2°, 11.9±0.2°, 12.6±0.2°, 13.0±0.2°, 13.4±0.2°, 14.6±0.2°, 15.0±0.2°, 15.6±0.2°, 16.0±0.2°, 16.8±0.2°, 17.5±0.2°, 17.8±0.2°, 18.0±0.2°, 19.4±0.2°, 19.9±0.2°, 20.2±0.2°, 20.8±0.2°, 23.0±0.2°, 23.7±0.2°, 25.4±0.2° and 26.2±0.2°.

In an embodiment, the XRPD pattern of the crystal form C is as shown in FIG. 10.

In a specific embodiment, the XRPD pattern analysis data of the crystal form C is as shown in Table 3.

TABLE 3
XRPD pattern analysis data of crystal form C

| | 2θ | Interplanar | Relative |
|---|---|---|---|
| Number | angle(°) | distance (Å) | intensity(%) |
| 1 | 6.7 | 13.27696 | 27.7 |
| 2 | 10.0 | 8.82743 | 53.1 |
| 3 | 10.6 | 8.35268 | 18.4 |
| 4 | 11.2 | 7.90886 | 18.1 |
| 5 | 11.9 | 7.46167 | 23.5 |
| 6 | 12.6 | 7.07054 | 17.9 |
| 7 | 13.0 | 6.86630 | 17.9 |
| 8 | 13.4 | 6.64174 | 17.9 |
| 9 | 14.6 | 6.10887 | 21.3 |
| 10 | 15.0 | 5.94183 | 29.8 |
| 11 | 15.6 | 5.73173 | 51.0 |
| 12 | 16.0 | 5.57620 | 15.0 |
| 13 | 16.8 | 5.32215 | 25.6 |
| 14 | 17.5 | 5.12446 | 16.6 |
| 15 | 17.8 | 5.02590 | 22.1 |
| 16 | 18.0 | 4.97285 | 16.0 |
| 17 | 18.9 | 4.76233 | 9.8 |
| 18 | 19.4 | 4.65028 | 37.4 |
| 19 | 19.9 | 4.52928 | 31.2 |
| 20 | 20.2 | 4.45318 | 100.0 |
| 21 | 20.8 | 4.32964 | 37.1 |
| 22 | 21.5 | 4.19610 | 11.3 |
| 23 | 22.5 | 4.02827 | 11.9 |
| 24 | 22.7 | 3.99511 | 14.6 |
| 25 | 23.0 | 3.94754 | 17.1 |
| 26 | 23.4 | 3.87846 | 13.6 |
| 27 | 23.7 | 3.83385 | 23.1 |
| 28 | 24.0 | 3.78645 | 14.6 |
| 29 | 24.6 | 3.69726 | 10.0 |
| 30 | 25.0 | 3.64450 | 8.0 |
| 31 | 25.4 | 3.58645 | 15.3 |
| 32 | 26.2 | 3.48764 | 19.5 |
| 33 | 27.1 | 3.37954 | 9.9 |
| 34 | 27.7 | 3.31590 | 7.2 |
| 35 | 28.5 | 3.23016 | 6.3 |
| 36 | 29.4 | 3.14036 | 7.5 |
| 37 | 29.8 | 3.10379 | 11.9 |
| 38 | 30.1 | 3.06819 | 6.4 |
| 39 | 31.0 | 2.98949 | 8.8 |
| 40 | 31.6 | 2.94143 | 8.2 |
| 41 | 32.2 | 2.88886 | 8.1 |
| 42 | 32.8 | 2.84453 | 6.6 |
| 43 | 33.3 | 2.80279 | 5.5 |
| 44 | 33.9 | 2.76440 | 6.6 |
| 45 | 35.5 | 2.65230 | 6.4 |
| 46 | 36.9 | 2.56808 | 4.9 |
| 47 | 37.1 | 2.55250 | 5.5 |
| 48 | 37.6 | 2.52207 | 5.5 |
| 49 | 39.3 | 2.43207 | 5.7 |
| 50 | 40.8 | 2.35952 | 5.4 |
| 51 | 41.2 | 2.34030 | 5.5 |
| 52 | 43.1 | 2.25529 | 7.2 |

In an embodiment, the differential scanning calorimetry curve (DSC) of the crystal form C of the citrate salt of the compound of formula (I) of the present invention has an endothermic peak at 194.3±3° C.

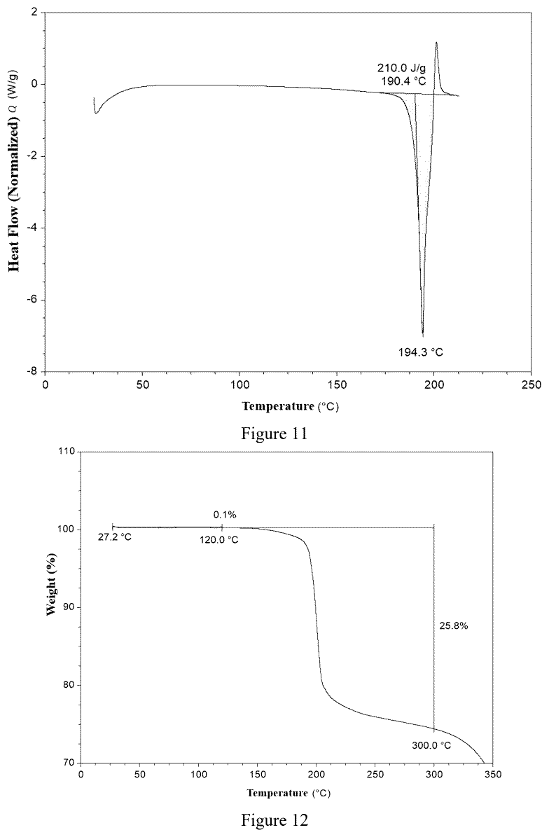

In an embodiment, the DSC spectrum of the crystal form C of the citrate salt of the compound of formula (I) of the present invention is as shown in FIG. 11.

In an embodiment, the thermogravimetric analysis curve (TGA) of the crystal form C of the citrate salt of the compound of formula (I) of the present invention has a weight loss of 0.1% at 120±3° C. In an embodiment, the thermogravimetric analysis curve (TGA) of the crystal form C of the citrate salt of the compound of formula (I) of the present invention has a weight loss of 25.8% at 120-300±3° C.

In an embodiment, the TGA spectrum of the crystal form C of the citrate salt of the compound of formula (I) of the present invention is as shown in FIG. 12.

### Crystal Form D

In another aspect, provided is a crystal form D of a citrate salt of the compound of formula (I).

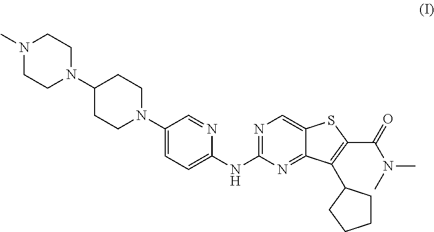

In an embodiment, in the crystal form D of the citrate salt of the compound of formula (I) of the present invention, the molar ratio of the compound to citric acid is about 1:1.

In an embodiment, the crystal form D of the citrate salt of the compound of formula (I) of the present invention is a methanol solvate of the citrate salt of the compound of formula (I).

In an embodiment, in the crystal form D of the citrate salt of the compound of formula (I) of the present invention, the molar ratio of the citrate salt of the compound of formula (I) to methanol is about 1:1.

In another embodiment, the X-ray powder diffraction pattern of the crystal form D of the citrate salt of the compound of formula (I) of the present invention has characteristic diffraction peaks at the following 2θ angles: 14.0±0.2°, 16.8±0.2°, 18.6±0.2°, 19.8±0.2° and 24.6±0.2°.

In a preferred embodiment, the X-ray powder diffraction pattern of the crystal form D of the citrate salt of the compound of formula (I) of the present invention has characteristic diffraction peaks at the following 2θ angles: 6.0±0.2°, 7.1±0.2°, 14.0±0.2°, 15.4±0.2°, 16.8±0.2°, 18.2±0.2°, 18.6±0.2°, 19.6±0.2°, 19.8±0.2°, 23.3±0.2°, 24.6±0.2° and 28.1±0.2°.

In a more preferred embodiment, the X-ray powder diffraction pattern of the crystal form D of the citrate salt of the compound of formula (I) of the present invention has characteristic diffraction peaks at the following 2θ angles: 6.0±0.2°, 7.1±0.2°, 9.2±0.2°, 11.3±0.2°, 12.1±0.2°, 14.0±0.2°, 15.4±0.2°, 15.7±0.2°, 16.8±0.2°, 17.4±0.2°, 18.2±0.2°, 18.6±0.2°, 19.0±0.2°, 19.6±0.2°, 19.8±0.2°, 21.0±0.2°, 21.2±0.2°, 22.4±0.2°, 22.7±0.2°, 23.3±0.2°, 23.5±0.2°, 23.7±0.2°, 24.6±0.2°, 28.1±0.2° and 28.8±0.2°.

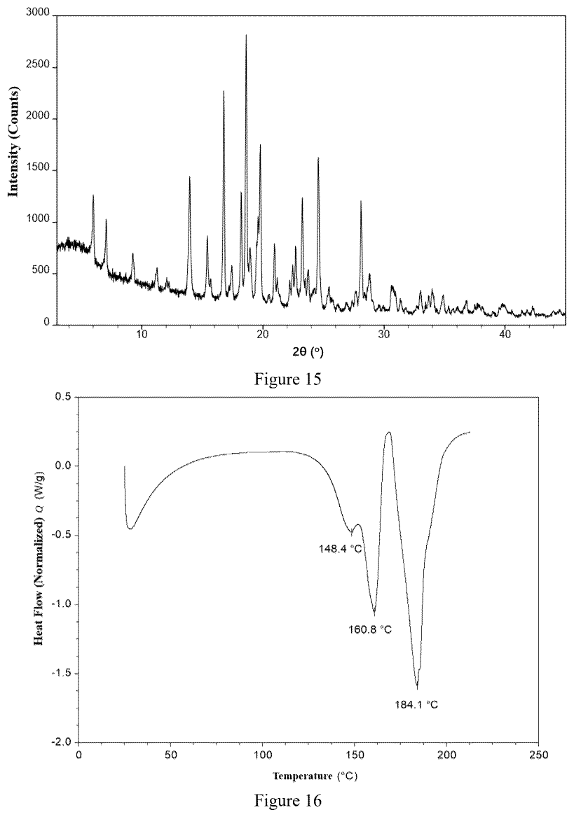

In an embodiment, the XRPD pattern of the crystal form D is as shown in FIG. 15.

In a specific embodiment, the XRPD pattern analysis data of the crystal form D is shown in Table 4.

TABLE 4
XRPD pattern analysis data of crystal form D

| | 2θ | Interplanar | Relative |
|---|---|---|---|
| Number | angle(°) | distance (Å) | intensity(%) |
| 1 | 6.0 | 14.72185 | 44.9 |
| 2 | 7.1 | 12.53934 | 36.5 |
| 3 | 9.2 | 9.58425 | 24.5 |
| 4 | 11.3 | 7.88158 | 19.9 |
| 5 | 12.1 | 7.36570 | 16.5 |
| 6 | 14.0 | 6.38435 | 51.2 |
| 7 | 15.4 | 5.79360 | 30.9 |
| 8 | 15.7 | 5.68969 | 16.1 |
| 9 | 16.8 | 5.33833 | 80.7 |
| 10 | 17.4 | 5.14315 | 20.6 |
| 11 | 18.2 | 4.93124 | 46.0 |
| 12 | 18.6 | 4.82381 | 100.0 |
| 13 | 19.0 | 4.74326 | 26.8 |
| 14 | 19.6 | 4.59336 | 36.1 |
| 15 | 19.8 | 4.54946 | 62.3 |
| 16 | 20.5 | 4.39317 | 10.7 |
| 17 | 21.0 | 4.30632 | 28.2 |
| 18 | 21.2 | 4.26049 | 15.6 |
| 19 | 22.2 | 4.07799 | 14.2 |
| 20 | 22.4 | 4.03497 | 18.3 |
| 21 | 22.7 | 3.99073 | 26.4 |
| 22 | 23.3 | 3.90119 | 43.9 |
| 23 | 23.5 | 3.86210 | 16.2 |
| 24 | 23.7 | 3.82785 | 19.1 |
| 25 | 24.3 | 3.74983 | 12.7 |
| 26 | 24.6 | 3.70281 | 57.9 |
| 27 | 25.4 | 3.58473 | 13.2 |
| 28 | 25.8 | 3.54383 | 8.4 |
| 29 | 26.2 | 3.48764 | 6.7 |
| 30 | 27.0 | 3.39779 | 7.2 |
| 31 | 27.4 | 3.35256 | 8.2 |
| 32 | 27.7 | 3.31880 | 11.6 |
| 33 | 28.1 | 3.27028 | 43.0 |
| 34 | 28.4 | 3.24111 | 11.1 |
| 35 | 28.8 | 3.19513 | 18.0 |
| 36 | 29.1 | 3.16747 | 8.2 |
| 37 | 29.6 | 3.12132 | 7.3 |
| 38 | 29.9 | 3.08648 | 6.8 |
| 39 | 30.7 | 3.01713 | 12.5 |
| 40 | 31.4 | 2.95910 | 8.3 |
| 41 | 31.8 | 2.92186 | 5.5 |
| 42 | 32.7 | 2.85367 | 6.6 |
| 43 | 33.0 | 2.82546 | 12.1 |
| 44 | 33.4 | 2.79501 | 8.2 |
| 45 | 33.7 | 2.77769 | 9.8 |
| 46 | 34.0 | 2.75687 | 12.7 |
| 47 | 34.9 | 2.69488 | 9.7 |
| 48 | 35.3 | 2.66343 | 5.8 |
| 49 | 35.7 | 2.64044 | 5.6 |
| 50 | 36.1 | 2.61548 | 5.8 |
| 51 | 36.8 | 2.57122 | 8.3 |
| 52 | 37.8 | 2.51238 | 7.2 |
| 53 | 39.0 | 2.44786 | 4.4 |
| 54 | 39.8 | 2.40656 | 6.6 |
| 55 | 40.6 | 2.36835 | 5.1 |
| 56 | 41.4 | 2.32933 | 5.1 |
| 57 | 41.8 | 2.31018 | 5.1 |
| 58 | 42.4 | 2.28685 | 5.9 |

In an embodiment, the differential scanning calorimetry curve of the crystal form D of the citrate salt of the compound of formula (I) of the present invention has an endothermic peak at 148.4±3° C., 160.8° C. and 184.1° C., respectively.

In an embodiment, the DSC spectrum of the crystal form D of the citrate salt of the compound of formula (I) of the present invention is as shown in FIG. 16.

In an embodiment, the thermogravimetric analysis curve (TGA) of the crystal form D of the citrate salt of the compound of formula (I) of the present invention has a weight loss of 0.6% at 110±3° C. In an embodiment, the thermogravimetric analysis curve (TGA) of the crystal form D of the citrate salt of the compound of formula (I) of the present invention has a weight loss of 1.7% at 110-150±3° C. In an embodiment, the thermogravimetric analysis curve (TGA) of the crystal form D of the citrate salt of the compound of formula (I) of the present invention has a weight loss of 27.7% at 150-300±3° C.

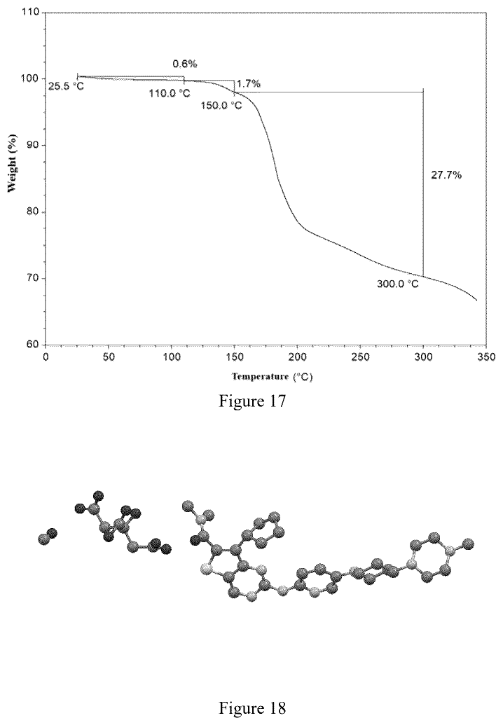

In an embodiment, the TGA spectrum of the crystal form D of the citrate salt of the compound of formula (I) of the present invention is as shown in FIG. 17.

### Preparation Process

### Preparation Process of Crystal Form A

In another aspect, provided is a process for preparing a crystal form A of a citrate salt of the compound of formula (I), comprising the following steps:
providing a solution of citric acid;
providing a solution of the compound of formula (I);
adding the solution of citric acid to the solution of the compound of formula (I);
stirring the obtained solution and cooling;
filtering the cooled solution and drying the filtrate to obtain the crystal form A of the compound of formula (I).

In a specific embodiment, the citrate salt of the compound of formula (I) is a dicitrate salt of the compound of formula (I).

In a specific embodiment, the solvent of the solution of citric acid is an alcohol solvent. In a specific embodiment, the solvent of the solution of citric acid is an ether solvent. In a specific embodiment, the solvent of the solution of citric acid is a nitrile solvent. In a specific embodiment, the solvent of the solution of citric acid is a ketone solvent.

In a specific embodiment, the solvent of the solution of the compound of formula (I) is an alcohol solvent. In a specific embodiment, the solvent of the solution of the compound of formula (I) is an ether solvent. In a specific embodiment, the solvent of the solution of the compound of formula (I) is a nitrile solvent. In a specific embodiment, the solvent of the solution of the compound of formula (I) is a ketone solvent.

In a preferred embodiment, the alcohol solvent is selected from the group consisting of methanol, ethanol, isopropanol, n-propanol, n-butanol, and a combination thereof. In a more preferred embodiment, the alcohol solvent is selected from the group consisting of methanol, ethanol, isopropanol, and a combination thereof. For example, methanol, ethanol or n-propanol.

In a preferred embodiment, the ether solvent is selected from the group consisting of ethyl ether, ethylene glycol methyl ether, ethylene glycol dimethyl ether, and a combination thereof. In a more preferred embodiment, the ether solvent is selected from the group consisting of ethylene glycol methyl ether, ethylene glycol dimethyl ether, and a combination thereof.

In a preferred embodiment, the nitrile solvent is selected from the group consisting of acetonitrile, propionitrile, butyronitrile and a combination thereof. In a more preferred embodiment, the nitrile solvent is acetonitrile.

In a preferred embodiment, the ketone solvent is selected from the group consisting of acetone, butanone, 4-methyl-2-pentanone, and a combination thereof. In a more preferred embodiment, the ketone solvent is selected from the group consisting of acetone, 4-methyl-2-pentanone, and a combination thereof.

In the process for preparing the crystal form A of the present invention, a suitable alcohol solvent is advantageous for obtaining the crystal form A of the present invention.

In a specific embodiment, a solution of the compound of formula (I) is heated. In a preferred embodiment, the solution of the compound of formula (I) is heated and the internal temperature of the solution is controlled to be 50-55° C.

In a specific embodiment, after heating and controlling the internal temperature of the solution of the compound of formula (I) to 50-55° C., the obtained solution is further stirred, and solid precipitation can be observed.

In a specific embodiment, the mixed solution of the compound of formula (I) and citric acid is cooled to an internal temperature of 10° C. In a further embodiment, the internal temperature of the mixed solution of the compound of formula (I) and citric acid is controlled to 10±2° C., and the solution is stirred, and crystal precipitation can be observed.

In an embodiment, in the solution of citric acid, the molar ratio of the compound of formula (I) as added to citric acid is about 1:3-1:5. In a preferred embodiment, the molar ratio of the compound of formula (I) as added to citric acid is about 1:3-1:4. For example, about 1:3, about 1:3.5, about 1:4, about 1:5. In the process for preparing the crystal form A of the present invention, a suitable molar ratio of the compound of formula (I) to citric acid is advantageous to obtain the crystal form A of the present invention.

### Preparation Process of Crystal Form B

In another aspect, provided is a process for preparing a crystal form B of a citrate salt of the compound of formula (I), comprising the following steps:
providing a solution of citric acid;
providing a solution of the compound of formula (I);
adding the solution of citric acid to the solution of the compound of formula (I);
stirring the obtained solution and cooling;
filtering the cooled solution and drying the filtrate to obtain the crystal form B of the compound of formula (I).

In a specific embodiment, the citrate salt of the compound of formula (I) is a dicitrate salt of the compound of formula (I).

In an embodiment, the solvent of the solution is selected from the group consisting of an alcohol solvent, water, and a combination thereof.

In a specific embodiment, the solvent of the solution of citric acid is an alcohol solvent.

In a specific embodiment, the solvent of the solution of the compound of formula (I) is an alcohol solvent and water.

In an embodiment, the alcohol solvent is selected from the group consisting of methanol, ethanol, isopropanol, n-propanol, and a combination thereof. In a preferred embodiment, the alcohol solvent is selected from the group consisting of methanol, ethanol, and a combination thereof. For example, methanol, ethanol or n-propanol.

In the process for preparing the crystal form B of the present invention, a suitable alcohol solvent is advantageous for obtaining the crystal form B of the present invention.

In a preferred embodiment, in the solution of the compound of formula (I), the mass ratio of the alcohol solvent to water is about 10:1-3:1. In a more preferred embodiment, in step (2), the mass ratio of the alcohol solvent to water is about 6:1-4:1. For example, about 10:1, about 9:1, about 8:1, about 7:1, about 6:1, about 5:1, about 4:1, about 3:1. In the process for preparing the crystal form B of the present invention, a suitable molar ratio of the compound of formula (I) to citric acid is advantageous to obtain the crystal form B of the present invention.

In a specific embodiment, a solution of the compound of formula (I) is heated. In a preferred embodiment, the solution of the compound of formula (I) is heated and the internal temperature of the solution is controlled to be 50-55° C.

In a specific embodiment, after heating and controlling the internal temperature of the solution of the compound of formula (I) to 50-55° C., the obtained solution is further stirred, and solid precipitation can be observed.

In a specific embodiment, the mixed solution of the compound of formula (I) and citric acid is cooled to an internal temperature of 10° C. In a further embodiment, the internal temperature of the mixed solution of the compound of formula (I) and citric acid is controlled to 10±2° C., and the solution is stirred, and crystal precipitation can be observed.

In an embodiment, in the solution of citric acid, the molar ratio of the compound of formula (I) as added to citric acid is about 1:3-1:5. In a preferred embodiment, the molar ratio of the compound of formula (I) as added to citric acid is about 1:3-1:4. For example, about 1:3, about 1:4, about 1:5. In the process for preparing the crystal form B of the present invention, a suitable molar ratio of the compound of formula (I) to citric acid is advantageous to obtain the crystal form B of the present invention.

### Preparation Process of Crystal Form C

In another aspect, provided is a process for preparing a crystal form C of a citrate salt of the compound of formula (I), comprising the following steps:
adding a citrate salt of the compound of formula (I) to solvent X to obtain a solution;
adding solvent Y to the above solution and stirring at room temperature;
obtaining the crystal form C by centrifugation.

In a specific embodiment, the citrate salt of the compound of formula (I) is a dicitrate salt of the compound of formula (I).

In an embodiment, the solvent X is selected from the group consisting of dimethylformamide, ethylene glycol methyl ether, and a combination thereof.

In an embodiment, the solvent Y is selected from the group consisting of acetonitrile, ethyl acetate, methanol, ethanol, isopropanol, and a combination thereof.

In the process for preparing the crystal form C of the present invention, a suitable alcohol solvent is advantageous for obtaining the crystal form C of the present invention.

In a preferred embodiment, the volume ratio of solvent X to solvent Y is about 5:1-10:1. For example, about 5:1, about 6:1, about 7:1, about 8:1, about 9:1, about 10:1.

In a preferred embodiment, the stirring time of the solution is about 1-3 days. For example, 1 day, 2 days, or 3 days.

### Preparation Process of Crystal Form D

In an embodiment, the crystal form D is prepared by the preparation process of the crystal form C, and the solvent is an alcohol solvent. In a more preferred embodiment, the solvent is methanol.

### Pharmaceutical Compositions and Administration

In one aspect, provided is a pharmaceutical composition comprising one or more selected from the group consisting of (i) the citrate salt of the compound of formula (I) of the present invention; (ii) the crystal form A of the citrate salt of the compound of formula (I) of the present invention; (iii) the crystal form B of the citrate salt of the compound of formula (I) of the present invention; (iv) the crystal form C of the citrate salt of the compound of formula (I) of the present invention; (v) the crystal form D of the citrate salt of the compound of formula (I) of the present invention.

In one aspect, provided is a pharmaceutical composition comprising the citrate salt of the compound of formula (I), and one or more pharmaceutically acceptable carriers.

In another aspect, provided is a pharmaceutical composition comprising the crystal form A of the citrate salt of the compound of formula (I), and one or more pharmaceutically acceptable carriers.

In yet another aspect, provided is a pharmaceutical composition comprising the crystal form B of the citrate salt of the compound of formula (I), and one or more pharmaceutically acceptable carriers.

In yet another aspect, provided is a pharmaceutical composition comprising the crystal form C of the citrate salt of the compound of formula (I), and one or more pharmaceutically acceptable carriers.

In another aspect, provided is a pharmaceutical composition comprising the crystal form D of the citrate salt of the compound of formula (I), and one or more pharmaceutically acceptable carriers.

As used herein, the term “pharmaceutically acceptable carrier” refers to diluents, adjuvants, excipients, or vehicles administered together with an active ingredient, and which, within the scope of sound medical judgment, are suitable for contact with the tissues of humans and/or other animals without excessive toxicity, irritation, allergic response, or other problems or complications commensurate with a reasonable benefit/risk ratio.

The administration of the salt of the compound of the present invention or the crystal forms thereof in pure form or in the form of a suitable pharmaceutical composition may be carried out by any acceptable mode of administration of an agent providing similar use. The pharmaceutical composition of the present invention can be prepared by combining the salt of the compound of the present invention or the crystal form thereof with a suitable pharmaceutically acceptable carrier.

The pharmaceutical composition of the present invention can be manufactured by processes well known in the art, such as conventional mixing processes and the like.

Generally, satisfactory results can be achieved at daily doses of 0.001 to 100 mg/kg body weight, specifically from about 0.03 to 2.5 mg/kg body weight. A daily dose of a larger mammal, such as a human, can be from about 0.5 mg to about 2000 mg, or more specifically, from 0.5 mg to 1000 mg, in a convenient form, for example, in divided doses up to four times per day or in a sustained release form. Suitable unit dosage forms for oral administration contain from about 1 to 50 mg of the active ingredient.

Typical routes for administering the compounds of the present invention or pharmaceutical compositions thereof include, but are not limited to, oral, rectal, transmucosal, enteral administration, or topical, transdermal, inhalation, parenteral, sublingual, intravaginal, intranasal, intraocular, intraperitoneal, intramuscular, subcutaneous, intravenous administration.

In a preferred embodiment, the pharmaceutical composition is in oral form. For oral administration, the pharmaceutical composition can be formulated by mixing the active compound with pharmaceutically acceptable carriers, excipients and/or media well known in the art. The pharmaceutical compositions of the present invention can be made by mixing, granulating, coating, dissolving or freeze-drying procedures in a conventional manner. These carriers, excipients and media enable the compounds of the present invention to be formulated into tablets, pills, lozenges, dragees, capsules, liquids, gels, slurries, suspensions and the like, for oral administration to patients. For example, the pharmaceutical composition comprises a compound of the present invention in combination with at least one pharmaceutically acceptable carrier or diluent, and can be prepared by mixing with a pharmaceutically acceptable carrier or diluent in a conventional manner. Unit dosage forms for oral administration include, for example, from about 0.1 mg to about 500 mg of active substance.

In an embodiment, the pharmaceutical composition is a solution of an active ingredient comprising a suspension or dispersion, such as an isotonic aqueous solution. In the case of a lyophilized composition comprising only the active ingredient or mixed with a carrier such as mannitol, the dispersion or suspension may be replenished prior to use. Pharmaceutical compositions may be sterilized and/or contain adjuvants such as preservatives, stabilizers, wetting agents or emulsifiers, dissolution enhancers, salts and/or buffering agents that regulate osmotic pressure. Suitable preservatives include, but are not limited to, antioxidants such as ascorbic acid, microbicides such as sorbic acid or benzoic acid. Solutions or suspensions may also contain viscosifiers, including but not limited to sodium carboxymethylcellulose, carboxymethylcellulose, dextran, polyvinylpyrrolidone, gelatin, or solubilizers such as Tween 80 (polyoxyethylene (20) sorbitan monooleate).

Suspensions may contain, in oil, vegetable oils, synthetic or semi-synthetic oils, as oily ingredients, commonly used for injection purposes. Examples include liquid fatty acid esters containing long chain fatty acids having from 8 to 22 carbon atoms, or in some embodiments, from 12 to 22 carbon atoms as the acid component. Suitable liquid fatty acid esters include, but are not limited to, lauric acid, tridecanoic acid, myristic acid, pentadecanoic acid, palmitic acid, heptadecanoic acid, stearic acid, arachidic acid, behenic acid, or corresponding unsaturated acids, such as oleic acid, elaidic acid, erucic acid, brassidic acid, and linoleic acid, and may contain antioxidants, if desired, such as vitamin E, 3-carotene, or 3,5-di-tert-butylhydroxytoluene. The alcohol component of these fatty acid esters can have six carbon atoms and can be monovalent or multivalent, such as mono-, di-, or trivalent alcohols. Suitable alcohol components include, but are not limited to, methanol, ethanol, propanol, butanol, or pentanol or isomers thereof, ethylene glycol, and glycerol.

Other suitable fatty acid esters include, but are not limited to, ethyl oleate, isopropyl myristate, isopropyl palmitate, M2375 (polyoxyethylene glycerol), M1944 CS (unsaturated PEGylated glycerides by alcoholysis of almond oil and containing glycerides and polyethylene glycol esters), LABRASOL™ (saturated PEGylated glycerides prepared by alcoholysis of TCM, and containing glycerides and polyethylene glycol esters; all available from GaKefosse, France), and/or 812 (saturated fatty acid triglycerides with a chain length of C8 to C12 from Hüls AG, Germany), and vegetable oils such as cottonseed oil, almond oil, olive oil, castor oil, sesame oil, soybean oil, or peanut oil.

In an embodiment, the solid oral composition can be prepared by conventional mixing, filling, or tableting processes. For example, it can be obtained by mixing the active compound with a solid excipient, optionally milling the resulting mixture, adding other suitable adjuvants if necessary, and then processing the mixture into granules to obtain the core of the tablet or dragee.

Suitable carriers include, but are not limited to, fillers such as sugars, cellulose preparations and/or calcium phosphate, binders, and/or disintegrating agent if necessary. Additional excipients include flow regulators and lubricants.

The tablet core may be provided with a suitable, optionally enteric coating, or a coating solution dissolved in a suitable organic solvent or solvent mixture, or, for enteric coatings. The dye or pigment may be added to the tablet or tablet coating.

Pharmaceutical compositions for oral administration may also include hard capsules, including gelatin or soft sealed capsules containing gelatin and a plasticizer, such as glycerol or sorbitol. Hard capsules may be in the form of granules of the active ingredient, for example, mixed with fillers such as corn starch, binders and/or glidants such as talc or magnesium stearate, and optionally stabilizers. In soft capsules, the active ingredient may be dissolved or suspended in a suitable liquid excipient such as fatty oil, paraffin oil or liquid polyethylene glycol, or fatty acid esters of ethylene glycol or propylene glycol, to which stabilizers and detergents, such as fatty acid esters of polyoxyethylene sorbitol, may also be added.

Pharmaceutical compositions suitable for rectal administration, such as suppositories, comprise a combination of the active ingredient and a suppository base. Suitable suppository bases are, for example, natural or synthetic triglycerides, paraffinic hydrocarbons, polyethylene glycols or higher alkanols.

Pharmaceutical compositions suitable for parenteral administration may comprise active ingredients in water-soluble form, such as water-soluble salts or aqueous injection suspensions comprising viscosity increasing substances, such as sodium carboxymethylcellulose, aqueous solutions of sorbitol and/or dextran if necessary, and stabilizers. The active ingredient, optionally with excipients, may also be in a freeze-dried form and may be a solution prepared by adding a suitable solvent prior to parenteral administration. Solutions used, for example, for parenteral administration, may also be used as infusion solutions. Injection formulations are typically prepared under aseptic conditions, filled, e.g., into ampoules or vials, and sealed containers.

The compounds of the present invention may be administered as the sole active ingredient, or together with other drugs useful in immunoregulatory therapy or antitumor diseases. For example, the compounds of the present invention are used with pharmaceutical compositions effective for the various diseases described above, for example, the compounds of the present invention can be used with aromatase inhibitors (e.g., letrozole, anastrozole, exemestane); or also with selective estrogen receptor modulators (e.g., fulvestrant, tamoxifen); or also with gonadal hormone releasing hormone agonists (e.g. goserelin, leuprolide, triptorelin, busereline); or also with cyclophosphamide, 5-fluorouracil, fludarabine, gemcitabine, cisplatin, carboplatin, vincristine, vinblastine, etoposide, irinotecan, paclitaxel, docetaxel, rituximab, doxorubicin, gefitinib or imatinib; or also with cyclosporin, rapamycin, ascomycin or their immunosuppressive analogs such as cyclosporin A, cyclosporine G, FK-506, sirolimus and everolimus, glucocorticoids such as: prednisone, cyclophosphamide, azathioprine, methotrexate, gold salts, sulfasalazine, antimalarials, brequinar, leflunomide, mizoribine, mycophenolic acid, mycophenolic acid, phenolic acid ester and 15-deoxysperguanamycin, immunosuppressive monoclonal antibodies such as monoclonal antibody leukocyte receptors such as MHC, CD2, CD3, CD4, CD7, CD25, CD28, I CD40, CD45, CD58, CD80, CD86, CD152, CD137, CD154, ICOS, LFA-1, VLA-4 or their ligands or other immunomodulatory compounds such as CTLA41g.

### Pharmaceutical Use

In one aspect, provided is use of the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or the pharmaceutical composition of the present invention in the manufacture of a medicament for treating, ameliorating or preventing an abnormal cell proliferation.

In an embodiment, provided is a method for treating, ameliorating or preventing an abnormal cell proliferation, comprising administering to a subject in need thereof a therapeutically effective amount of the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or the pharmaceutical composition of the present invention.

In another embodiment, provided is the citrate salt of the compound of formula (I) of the present invention or the crystal form A of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or the pharmaceutical composition of the present invention, which is used for treating, ameliorating or preventing an abnormal cell proliferation.

In another aspect, provided is use of the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or the pharmaceutical composition of the present invention in the manufacture of a medicament for treating, ameliorating or preventing a condition responsive to inhibition of cyclin-dependent kinase 4/6.

In an embodiment, provided is a method for treating, ameliorating or preventing a condition responsive to inhibition of cyclin-dependent kinase 4/6, comprising administering to a subject in need thereof a therapeutically effective amount of the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or pharmaceutical composition of the present invention.

In another embodiment, provided is the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or the pharmaceutical composition of the present invention, which is used for treating, ameliorating or preventing a condition responsive to inhibition of cyclin-dependent kinase 4/6.

In an embodiment, the cell proliferative disease is selected from the group consisting of a cancerous proliferative disease (e.g., brain, lung, squamous cell, bladder, stomach, pancreas, breast, head, neck, kidney, ovary, prostate, colorectal, epidermis, esophagus, testis, gynecology or thyroid cancer); a non-cancerous proliferation disorder (e.g., benign skin hyperplasia (e.g., psoriasis), restenosis, and benign prostatic hypertrophy (BPH)); pancreatitis; kidney disease; pain; prevention of blastocyst landing; treatment of diseases associated with vasculogenesis or angiogenesis (e.g., tumor angiogenesis, acute and chronic inflammatory diseases such as rheumatoid arthritis, atherosclerosis, inflammatory bowel disease, skin disorders such as psoriasis, eczema and scleroderma, diabetes, diabetic retinopathy, retinopathy of prematurity, age-related macular degeneration, hemangioma, glioma, melanoma, Kaposi's sarcoma and ovarian cancer, breast cancer, lung cancer, pancreatic cancer, prostate cancer, colon cancer, and epidermoid cancer); asthma; neutrophil chemotaxis (e.g., myocardial infarction and stroke reperfusion injury and inflammatory arthritis); septic shock; T cell-mediated disease, where immunosuppression is valuable (e.g., prevention of organ transplant rejection, graft-versus-host disease, lupus erythematosus, multiple sclerosis, and rheumatoid arthritis); atherosclerosis; inhibition of keratinocytes responsive to growth factor mixtures; chronic obstructive pulmonary disease (COPD) and other diseases.

In a preferred embodiment, the cell proliferative disease is hormone receptor (HR), advanced or metastatic breast cancer negative in human epidermal growth factor receptor 2 (HER2).

In an embodiment, the 4/6 gene mutation causes the aforementioned disease, e.g., melanoma, lung cancer, colon cancer, etc.

### Combination Therapy

The citrate salt, the crystal forms A-D thereof or the composition of the compound of formula (I) of the present invention can be used alone or in combination with other therapeutic agents.

For example, the use of adjuvant drugs may enhance the therapeutic effect of the citrate salt of the compound of the present invention or the crystal form thereof (e.g., the therapeutic benefit of using the adjuvant drug alone is minimal, but can enhance the therapeutic benefit of the individual when used in combination with another drug), or, for example, the citrate salt of the compound of the present invention or the crystal form thereof may be used to enhance the therapeutic benefit of an individual with another therapeutic agent having the same therapeutic effect. For example, in the treatment of gout, the use of the citrate salt of the compound of the present invention or the crystal form thereof in combination with another drug for treating gout may enhance clinical benefits. Alternatively, for example, if the adverse reaction using the citrate salt of the compound of the present invention or the crystal form thereof is nausea, an anti-nausea drug can be used in combination. Alternatively, the combination therapy may include, but is not limited to, physical therapy, psychotherapy, radiation therapy, compression therapy of disease areas, rest and dietary improvement, etc. Regardless of the disease, discomfort, or symptoms, both therapies should have additive or synergistic effects that benefit the treatment of an individual.

In the case where the citrate salt of the compound of the present invention or the crystal form thereof is used in combination with other therapeutic agents, the administration route of the pharmaceutical composition of the compound of the present invention may be the same as that of other drugs, or the administration route may be different due to different physical and chemical properties. For example, oral administration of the citrate salt of the compound of the present invention or the crystal form thereof may produce and maintain a good blood drug level, while another therapeutic agent may require intravenous administration. Therefore, the citrate salt of the compound of the present invention or the crystal form thereof may be administered simultaneously, sequentially or separately with another therapeutic agent.

The citrate salt of the compound of formula (I) or the crystal form thereof is expected to be effective in combination with one or more of the following drugs: alkylating agents, angiogenesis inhibitors, antibodies, anti-metabolites, anti-mitotic, anti-hyperplasia, anti-viral agents, aurora kinase inhibitors, other apoptosis initiators (e.g., Bcl-xL, Bcl-w, and Bfl-1) inhibitors, death receptor pathway activators, Bcr-Abl kinase inhibitors, antibodies to BiTE (bispecific T-cell engager), antibody drug conjugates, biological response modifiers, cyclin-dependent kinase inhibitors, cell cycle inhibitors, cyclooxygenase-2 inhibitors, DVDs, leukemia viral oncogene cognate gene (ErbB2) receptor inhibitors, growth factor inhibitors, heat shock protein (HSP)-90 inhibitors, histone acetylase (HDAC) inhibitors, hormone therapy, immune agents, inhibitors of apoptosis protein inhibitors (IAPs), embedded antibiotics, kinase inhibitors, kinesin inhibitors, JAK2 inhibitors, rapamycin inhibitors against mammals, microRNAs, mitogen-activated extracellular signal-regulated kinase inhibitors, multivalent binding proteins, non-steroidal anti-inflammatory drugs (NSAIDs), polyADP (adenosine diphosphate)-ribose polymerase (PARP) inhibitors, platinum-based chemotherapeutic drugs, polo-like kinase (Plk) inhibitors, phosphoinositide 3 kinase (PI3K) inhibitors, proteasome inhibitors, purine analogs, pyrimidine analogs, receptor tyrosine kinase inhibitors, retinoid/deltoid plant alkaloids, small interfering RNAs (siRNAs), topoisomerase inhibitors, ubiquitin ligase inhibitors and analogs.

In an embodiment, provided is use of the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or the pharmaceutical composition of the present invention, optionally in combination with a second therapeutic agent, in the manufacture of a medicament for treating, ameliorating, or preventing a condition responsive to inhibition of cyclin dependent kinase 4/6.

In an embodiment, provided is a method for treating, ameliorating or preventing a condition responsive to inhibition of cyclin-dependent kinase 4/6, comprising administering to a subject in need thereof a therapeutically effective amount of the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or pharmaceutical composition of the present invention, and optionally a second therapeutic agent.

In an embodiment, provided is the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or the pharmaceutical composition of the present invention, optionally in combination with a second therapeutic agent, which is used for treating, ameliorating or preventing a condition that is responsive to inhibition of the cyclin-dependent kinase 4/6.

In an embodiment, provided is use of the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or the pharmaceutical composition of the present invention, optionally in combination with a second therapeutic agent, in the manufacture of a medicament for treating, ameliorating, or preventing an abnormal cell proliferation.

In an embodiment, provided is a method for treating, ameliorating or preventing an abnormal cell proliferation, comprising administering to a subject in need thereof a therapeutically effective amount of the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or the pharmaceutical composition of the present invention, and optionally a second therapeutic agent.

In an embodiment, provided is the citrate salt of the compound of formula (I) of the present invention or the crystal form A of the citrate salt of the compound of formula (I) of the present invention or the crystal form B of the citrate salt of the compound of formula (I) of the present invention or the crystal form C of the citrate salt of the compound of formula (I) of the present invention or the crystal form D of the citrate salt of the compound of formula (I) of the present invention or the pharmaceutical composition of the present invention, optionally in combination with a second therapeutic agent, which is used for treating, ameliorating or preventing an abnormal cell proliferation.

### Beneficial Effects

Compared with other salt forms, the citrate salt of the compound of formula (I) of the present invention has the beneficial effects of high stability and good solubility.

In addition, compared with other crystal forms or amorphous forms, the crystal form A, the crystal form B, the crystal form C, and the crystal form D of the citrate salt of the compound of formula (I) of the present invention also have excellent hygroscopicity, moisture absorption, and humidity stability, for example, under conditions of humidity cycling and high humidity, the crystal forms of the present invention do not undergo transformation, and exhibit excellent hygroscopicity, moisture absorption, and humidity stability. Further, compared with other crystalline forms or amorphous forms, the crystal form A, the crystal form B, and the crystal form C of the citrate salt of the compound of formula (I) of the present invention have significantly improved hygroscopicity, moisture absorption, and humidity stability, for example, under the conditions of humidity cycling and high humidity, the crystal form A, the crystal form B, and the crystal form C of the present invention do not undergo transformation, and exhibit significantly improved hygroscopicity, moisture absorption, and humidity stability.

In addition, compared with other crystal forms or amorphous forms, the crystal form A, the crystal form B, the crystal form C and the crystal form D of the citrate salt of the compound of formula (I) of the present invention also have excellent stability, such as high temperature stability and photostability. For example, under the conditions of high temperature and/or illumination, the crystal forms of the present invention do not undergo transformation, and exhibit excellent high temperature stability and photostability. Further, compared with other crystal forms or amorphous forms, the crystal form A, the crystal form B, and the crystal form C of the citrate salt of the compound of formula (I) of the present invention have significantly improved stability, such as high temperature stability and photostability. For example, under the conditions of high temperature and/or light, the crystal form A, the crystal form B and the crystal form C of the present invention will not be transformed, showing significantly improved high temperature stability and light stability.

Furthermore, compared with other crystal forms or amorphous forms, the crystal form A, the crystal form B, the crystal form C and the crystal form D of the citrate salt of the compound of formula (I) of the present invention have excellent solubility, for example, in biological media such as FaSSIF, FeSSIF and FaSSGF.

Based on this, the citrate salt of the compound of formula (I) of the present invention and the crystal form A, the crystal form B, the crystal form C and the crystal form D thereof have broad prospects for pharmaceutical development.

### EXAMPLES

The solutions of the present invention are further described in detail below with reference to specific embodiments.

It should be noted that the following embodiments are merely examples for clearly describing the technical solutions of the present invention, rather than limiting the present invention. It will be apparent to those skilled in the art that other different forms of variations or modifications may be made on the basis of the above description, and it is not necessary to exhaustively enumerate all the embodiments, and obvious variations or modifications derived therefrom still fall within the protection scope of the present invention. Unless otherwise specified, the instrumentation and reagent materials used herein are commercially available.

### High Resolution Mass Spectrometry (HRMS):

The mass spectra of the compound and salts and crystal forms thereof were detected by Agilent 1290 Infinity-6546 LC/Q-TOF. The ion source was ESI (+).

### Nuclear Magnetic Resonance Spectroscopy (NMR):

NMR spectra of the compound and salts and crystal forms were detected by BRUKER Av NEO 400M nuclear magnetic resonance spectrometer, wherein the solvent was D₂O or DMSO-d₆.

### X-Ray Powder Diffraction (XRPD):

The XRPD pattern of each crystal form was collected using a Panalytical EMPYREAN (Malvern Panalytical, UK) powder diffractometer, which was irradiated with Cu palladium, and continuous projection scanning was performed using Absolute scan at room temperature, with a scanning 20 range of 3 to 45°, a step size of 0.013°, and a single sample scanning duration of 5 min. When the sample was tested, the voltage and current of the light pipe were 45 kV and 40 mA, respectively, and the sample pan was a zero-background sample pan.

### Differential Scanning Calorimetry (DSC):

The DSC spectra of the crystal forms were collected by TA Discovery 250 (TA, US), the sample was heated to the final temperature at a rate of 10° C./min, the nitrogen purge rate at the sample was 60 mL/min, and the nitrogen purge rate at the balance was 40 mL/min.

### Thermogravimetric Analysis (TGA):

The TGA spectra of the crystal forms were collected by TA Discovery 550 (TA, US), the sample was heated to the final temperature at a rate of 10° C./min, the nitrogen purge rate at the sample was 60 mL/min, and the nitrogen purge rate at the balance was 40 mL/min.

### Dynamic Vapor Sorption and Desorption Analysis (DVS)

Dynamic vapor sorption and desorption analysis of crystal forms were performed using DVS Intrinsic (SMS, UK) to evaluate the hygroscopicity of the crystal forms. The gradient mode was used for the test. The humidity change was 50%-95%-50%. The humidity change amount of each gradient was 15%. The gradient endpoint was determined in the dm/dt manner, with dm/dt less than 0.002% and maintained for 10 minutes as the gradient endpoint, or the maximum maintenance time of each gradient was 60 min. After the test was completed, XRPD analysis was performed on the sample to confirm whether the solid form changed.

All solvents used herein are commercially available without further purification.

### Materials

The compound of formula (I) was prepared according to the procedures of CN109153686A.
Citric acid: purchased from General Reagent.
Methanol: purchased from General Reagent.
Dimethylformamide: purchased from General-Reagent.
4-Methyl-2-pentanone: purchased from General-Reagent.
Acetonitrile: purchased from General-Reagent.
Ethylene glycol dimethyl ether: purchased from General-Reagent.
Ethylene glycol methyl ether: purchased from General-Reagent.
Acetone: purchased from Guangzhou Chemical Reagent Factory.
FaSSIF: purchased from Biorelevant company, Batch number: FASBUF-1121-A.
FaSSGF: purchased from Biorelevant company, Batch number: FASBUF-1121-A.
FeFFIF: purchased from Biorelevant company, Batch number: FASBUF-0122-A.
Biological medium powder: purchased from Biorelevant company, Batch number: FFF-0521-A.

### PREPARATION EXAMPLES

### Example 1 Preparation of Citrate Salt of the Compound of Formula (I)

27.5 mg of the compound of formula (I) was added to 2.0 mL of solvent followed by citric acid added to the above solution. The obtained solution was suspended for 2 days at room temperature, and then the suspension was centrifuged to obtain the citrate salt of the present invention.

The solvent may be methanol or tetrahydrofuran.

When the feeding molar ratio of the compound of formula (I) to citric acid was about 1:3, dicitric acid salt of the compound of formula (I) was obtained, wherein the molar ratio of the compound of formula (I) to citric acid was 1:2.

When the feeding molar ratio of the compound of formula (I) to citric acid was about 1:1, a monocitrate salt of the compound of formula (I) was obtained, wherein the molar ratio of the compound of formula (I) to citric acid was 1:1.

wherein the characterization data of the dicitrate salt of the compound of formula (I) was as follows:

¹HNMR (400 MHz, DMSO-d₆) δ ppm 10.580 (brs, 1H), 9.681 (s, 1H), 9.681 (s, 1H), 8.042 (brs, 1H), 7.426 (brd, 1H), 8.140 (brd, 1H), 3.715 (brd, 2H), 3.149 (m, 1H), 3.149 (m, 2H), 3.043 (brs, 3H), 2.933 (brs, 3H), 2.933 (brs, 2H), 2.834 (brs, 2H), 2.648 (m, 3H), 2.648 (m, 2H), 2.648 (m, 2H), 2.648 (m, 2H), 2.211 (brs, 3H), 1.906 (brd, 2H), 1.906 (brd, 2H), 1.674 (m, 2H), 1.586 (m, 2H).

The characterization data of the monocitrate salt of the compound of formula (I) was as follows:

¹HNMR (400 MHz, DMSO-d₆) δ 9.53 (s, 1H), 9.16 (s, 1H), 8.11 (d, 1H), 8.01 (d, 1H), 7.40 (dd, 1H), 3.70 (br d, 2H), 3.03 (s, 3H), 2.92 (s, 3H), 2.67 (m, 10H), 2.19 (br d, 2H), 1.88 (br d, 6H), 1.66 (br d, 2H), 1.56 (m, 2H).

### Example 2 Preparation of Crystal Form A of Citrate Salt of the Compound of Formula (I)

(1) 23.8 g of anhydrous citric acid was dissolved in 100 g of methanol, and the obtained solution was used for later use.

(2) 300 g of methanol and 17 g of the compound of formula (I) were added to a 1000 ml reaction flask. The obtained solution was stirred and heated, and the internal temperature of the solution was controlled to be 50-55° C.

(3) To the solution of step (2) was added the methanol citrate solution of step (1) (i.e., the molar ratio of the compound of formula (I) to citric acid in the solution was 1:4), the internal temperature of the solution was controlled to be 50-55° C., and the solution was stirred for 15 min. During stirring, solid precipitation was observed.

(4) The mixed solution obtained in step (3) was cooled for 3 h to an internal temperature of 10° C.

(5) Under the condition that the internal temperature was 10±2° C., the mixed solution in step (4) was stirred for 1 h, and crystal precipitation was observed.

(6) The mixture obtained in step (5) was filtered, and the filtrate was dried under reduced pressure at 50° C. to obtain 27 g of the crystal form A of the citrate salt of the compound of formula (I) of the present application, with a yield of 93.4%. The crystal form A obtained was a light yellow powder, odorless, and slightly hygroscopic. The crystal form A was readily soluble in water, 0.1 mol/L hydrochloric acid solution, acetic acid, slightly soluble in methanol, and nearly insoluble in ethanol. The melting point of the crystal form A was 176.5° C.-180.5° C. pKa was 3.47, 4.86, and 8.18. ¹HNMR (400 MHz, DMSO-d₆) δ ppm 9.57 (s, 1H), 9.17 (s, 1H), 8.12 (d, 1H), 8.02 (d, 1H), 7.41 (dd, 1H), 3.72 (m, 2H), 3.17 (m, 2H), 3.04 (s, 4H), 2.93 (s, 3H), 2.80 (br s, 2H), 2.62 (m, 15H), 2.19 (m, 2H), 1.90 (m, 6H), 1.61 (m, 4H).

¹HNMR (400 MHz, D₂O) δ 9.00 (s, 1H), 7.80 (dd, 1H), 7.52 (d, 1H), 7.39 (d, 1H), 3.61 (m, 2H), 3.16 (m, 3H), 2.94 (s, 3H), 2.83 (s, 3H), 2.79 (m, 4H), 2.66 (m, 6H), 2.09 (br d, 2H), 1.84 (m, 6H), 1.63 (m, 4H).

Referring to FIG. 3, the TGA test results of the crystal form A showed that there was a weight loss of 1.2% during the heating of the crystal form A to 120° C., and a weight loss of 38.4% at 140-300° C. Referring to FIG. 2, the DSC test results of the crystal form A showed that there was an endothermic peak at 190° C. In addition, referring to FIG. 4, the NMR test results of the crystal form A in DMSO-do showed no obvious solvent peak, wherein the peak corresponding to citric acid at 2.53-2.73 ppm partially overlapped with the solvent peak of DMSO. The results of NMR test of the crystal form A in D₂O (referred to FIG. 5) showed that peaks at 2.80, 2.76, 2.68 and 2.64 ppm were represented characteristic peaks of citric acid. And it could be judged according to the integration thereof that in the crystal form A, the compound of formula (I): citric acid=1:2. Based on the above all the test results of crystal form, it could be seen that the crystal form A was dicitrate of the compound of formula (I), and the crystal form A did not contain crystallization water.

### Example 3 Preparation of Crystal Form B of Citrate Salt of the Compound of Formula (I)

(1) 23.8 g of anhydrous citric acid was dissolved in 100 g of methanol, and the obtained solution was used for later use.

(2) 300 g of methanol, 80 g of purified water and 17 g of the compound of formula (I) were added to a 1000 ml reaction flask. The obtained solution was stirred and heated, and the internal temperature of the solution was controlled to be 50-55° C.

(3) To the solution of step (2) was added the methanol citrate solution of step (1) (i.e., the molar ratio of the compound of formula (I) to citric acid in the solution was 1:4), the internal temperature of the solution was controlled to be 50-55° C., and the solution was stirred for 15 min. During stirring, solid precipitation was observed.

(4) The mixed solution obtained in step (3) was cooled for 3 h to an internal temperature of 10° C.

(5) Under the condition that the internal temperature was 10±2° C., the mixed solution in step (4) was stirred for 1 h, and crystal precipitation was observed.

(6) The mixture obtained in step (5) was filtered, and the filtrate was dried under reduced pressure at 50° C. to obtain 27 g of the crystal form B of the citrate salt of the compound of formula (I) of the present application, with a yield of 76.1%.

¹HNMR (400 MHz, D₂O) δ 9.04 (s, 1H), 7.86 (br d, 1H), 7.56 (s, 1H), 7.40 (d, 1H), 3.64 (br d, 2H), 3.05 (s, 3H), 2.94 (s, 3H), 2.79 (s, 3H), 2.75 (m, 3H), 2.65 (m, 6H), 2.08 (br d, 2H), 1.84 (m, 6H), 1.62 (m, 4H).

Referring to FIG. 8, the TGA test results of the crystal form B showed that there was a weight loss of 2.2% during the heating of the crystal form B to 90° C., a weight loss of 1.8% from 90° C. to 140° C., and a weight loss of 36.9% from 140° C. to 300° C. It could be seen that when the crystal form B was heated to 140° C., the crystal form B had a total weight loss of 4%, which was equivalent to the theoretical water content (3.7%) of the dihydrate. In addition, referring to FIG. 7, the DSC test results of the crystal form B showed that the crystal form B had endothermic peaks at about 81, 133, 151, and 178° C. Based on the above test results and the NMR data of the crystal form B in D₂O, it could be seen that the crystal form B contained crystallization water, and was the hydrate of the dicitrate salt of the compound of formula (I), and in the crystal form B, the molar ratio of the citrate salt of the compound of formula (I) to the crystallization water was about 1:2.

### Example 5 Preparation of Crystal Form C of Citrate Salt of the Compound of Formula (I)

(1) 99.9 mg of dicitrate salt of the compound of (I) was added to a glass vial;

(2) Dimethylformamide or ethylene glycol methyl ether or trifluoroethanol was added to the container of step (1);

(3) Acetonitrile or ethyl acetate or methanol or ethanol or isopropanol was added to the solution of step (2);

(4) The mixture obtained in step (3) was stirred at room temperature for 1-2 days;

(5) The mixture obtained in step (4) was separated by centrifugation to obtain the crystal form C of the compound of formula (I).

When the solvent added in step (2) was dimethylformamide, the solvent in step (3) could be ethanol or isopropanol or acetonitrile. When the solvent added in step (2) is trifluoroethanol, the solvent in step (3) could be ethanol. When the solvent added in step (2) is ethylene glycol methyl ether, the solvent in step (2) could be ethyl acetate.

¹HNMR (400 MHz, DMSO-d₆) δ 9.53 (s, 1H), 9.16 (s, 1H), 8.11 (d, 1H), 8.01 (d, 1H), 7.40 (dd, 1H), 3.70 (br d, 2H), 3.03 (s, 3H), 2.92 (s, 3H), 2.67 (m, 10H), 2.19 (br d, 2H), 1.88 (br d, 6H), 1.66 (br d, 2H), 1.56 (m, 2H).

¹HNMR (400 MHz, D₂O) δ 8.94 (s, 1H), 7.62 (br s, 1H), 7.58 (br s, 1H), 7.53 (m, 1H), 3.53 (m, 2H), 3.10 (s, 3H), 2.75 (m, 5H), 2.62 (m, 5H), 1.99 (br d, 2H), 1.84 (br s, 4H), 1.73 (br s, 2H), 1.61 (br s, 2H), 1.53 (br d, 2H).

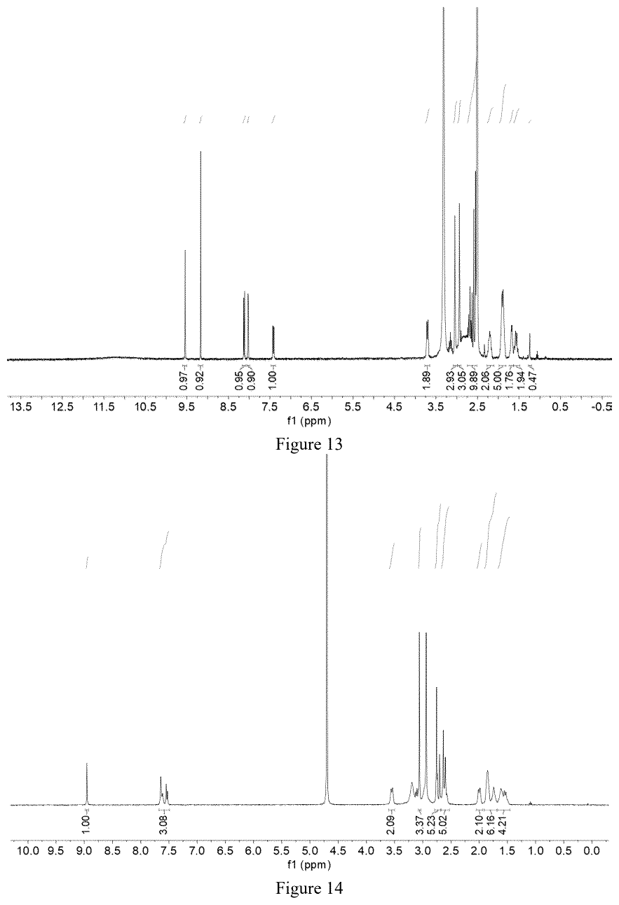

Referring to FIG. 12, the TGA test results of the crystal form C showed that there was a weight loss of 0.1% during the heating of the crystal form C to 120° C., and a weight loss of 25.8% at 120-300° C. In addition, referring to FIG. 11, the DSC test results of the crystal form C showed that at about 194° C., the crystal form C had endothermic peak corresponding to melting accompanied by decomposition. In addition, referring to FIG. 13, the NMR test results of crystal form C in DMSO-do showed that the integration result at 2.54-2.75 ppm was different from the compound of formula (I). In addition, referring to FIG. 14, the NMR test results of the crystal form C in D₂O showed that the characteristic peaks at 2.74, 2.70, 2.64 and 2.60 ppm corresponded to citric acid, and the integration results showed that in the crystal form C, the compound of formula (I): citric acid=1:1. Based on the above test results, the crystal form C was the monocitrate salt of the compound of formula (I), and the crystal form C did not contain crystallization water.

### Example 6 Preparation of Crystal Form D of Citrate Salt of the Compound of Formula (I)

The preparation procedures of the crystal form D of the citrate salt of the compound of formula (I) was similar to that of the crystal form C, except that the solvent added in step (2) was dimethylformamide, and the solvent added in step (3) was methanol.

¹HNMR (400 MHz, DMSO-d₆) δ 9.56 (s, 1H), 9.16 (s, 1H), 8.12 (d, 1H), 8.02 (d, 1H), 7.41 (dd, 1H), 4.09 (br s, 1H), 3.71 (br d, 2H), 2.61 (m, 13H), 2.19 (br d, 2H), 1.90 (m, 6H), 1.67 (br d, 2H), 1.57 (m, 2H).

Referring to FIG. 18, the single crystal diagram of the crystal form D showed that the crystal form D was a methanol solvate of the monocitrate salt of the compound of formula (I).

In addition, according to the single crystal diagram of FIG. 18, it could be seen that in the crystal form D, the ratio of the citrate salt of the compound of formula (I) to methanol was 1:1.

### Comparative Example Preparation of Other Salt Forms of the Compound of Formula (I)

27.5 mg of the compound of formula (I) was added to 2.0 mL of solvent followed by acid addition to the above solution. The obtained solution was suspended for 2 days at room temperature, and then the suspension was centrifuged to obtain the salt of the present invention.

### Comparative Example 1 Hydrochloride Salt of the Compound of Formula (I)

When the added acid was hydrochloric acid, the hydrochloride salt of the compound of formula (I) was obtained.

### Comparative Example 2 Sulfate Salt of the Compound of Formula (I)

When the added acid was sulfuric acid, the sulfate salt of the compound of formula (I) was obtained.

### Comparative Example 3 Hydrobromide Salt of the Compound of Formula (I)

When the added acid was hydrobromic acid, the hydrobromide salt of the compound of formula (I) was obtained.

### Comparative Example 4 Mesylate Salt of the Compound of Formula (I)

When the added acid was methanesulfonic acid, mesylate salt of the compound of formula (I) was obtained.

### Comparative Example 5 p-Tosylate Salt of the Compound of Formula (I)

When the added acid was p-toluenesulfonic acid, p-tosylate salt of the compound of formula (I) was obtained.

### Comparative Example 6 Maleate Salt of the Compound of Formula (I)

When the added acid was maleic acid, the maleate salt of the compound of formula (I) was obtained.

### Comparative Example 7 Fumarate Salt of the Compound of Formula (I)

When the added acid was fumaric acid, the fumarate salt of the compound of formula (I) was obtained.

### Comparative Example 8 Oxalate Salt of the Compound of Formula (I)

When the added acid was oxalic acid, the oxalate salt of the compound of formula (I) was obtained.

### Comparative Example 9 L-Tartrate Salt of the Compound of Formula (I)

When the added acid was L-tartaric acid, the L-tartrate salt of the compound of formula (I) was obtained.

### Comparative Example 10 Succinate Salt of the Compound of Formula (I)

When the added acid was succinic acid, the succinate salt of the compound of formula (I) was obtained.

### Comparative Example Preparation of Other Crystal Forms of the Compound of Formula (I)

### Comparative Example a Crystal Form E of the Compound of Formula (I)

The crystal form E of the compound of formula (I) could be obtained by heating the in-situ thermal stage of the crystal form B of the present invention to 81° C.

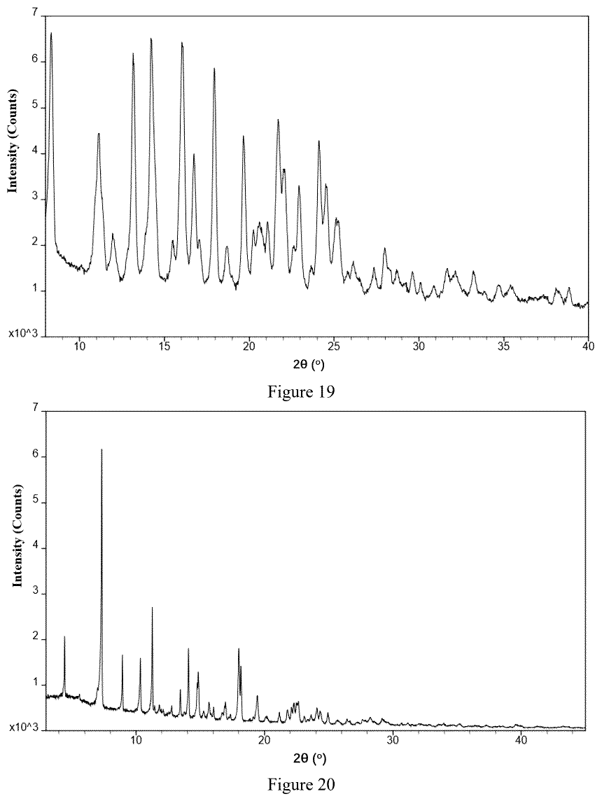

The XRPD pattern of the crystal form E was as shown in FIG. 19.

The XRPD pattern data of the crystal form E was shown in the following table:

TABLE 5
XRPD pattern analysis data of crystal form E

| | 2θ | Interplanar | Relative |
|---|---|---|---|
| Number | angle(°) | distance (Å) | intensity(%) |
| 1 | 8.4 | 10.61397 | 100.0 |
| 2 | 11.1 | 7.98255 | 66.9 |
| 3 | 12.0 | 7.42496 | 33.7 |
| 4 | 13.2 | 6.76144 | 93.3 |
| 5 | 14.2 | 6.27433 | 98.2 |
| 6 | 15.5 | 5.77424 | 31.7 |
| 7 | 16.0 | 5.57588 | 96.9 |
| 8 | 16.8 | 5.34365 | 60.3 |
| 9 | 17.1 | 5.23816 | 30.8 |
| 10 | 18.0 | 4.99843 | 88.4 |
| 11 | 19.7 | 4.57720 | 66.2 |
| 12 | 20.3 | 4.44994 | 35.3 |
| 13 | 20.6 | 4.37802 | 37.9 |
| 14 | 21.1 | 4.28309 | 37.9 |
| 15 | 21.7 | 4.16452 | 71.6 |
| 16 | 22.0 | 4.10976 | 55.2 |
| 17 | 22.6 | 4.01210 | 29.6 |
| 18 | 22.9 | 3.95447 | 49.8 |
| 19 | 23.6 | 3.84111 | 23.6 |
| 20 | 24.1 | 3.76940 | 64.6 |
| 21 | 24.6 | 3.70671 | 49.5 |
| 22 | 25.3 | 3.60816 | 38.2 |
| 23 | 25.8 | 3.54006 | 21.8 |
| 24 | 26.2 | 3.49623 | 25.0 |
| 25 | 27.4 | 3.35195 | 22.9 |
| 26 | 28.0 | 3.28238 | 29.4 |
| 27 | 28.3 | 3.24996 | 22.1 |
| 28 | 28.7 | 3.20711 | 21.7 |
| 29 | 29.6 | 3.11685 | 21.6 |
| 30 | 30.1 | 3.06986 | 18.0 |
| 31 | 30.9 | 3.00145 | 17.0 |
| 32 | 31.6 | 2.93653 | 22.3 |
| 33 | 32.1 | 2.89565 | 21.8 |
| 34 | 33.2 | 2.80968 | 21.4 |
| 35 | 33.9 | 2.76348 | 14.7 |
| 36 | 34.7 | 2.70717 | 17.2 |
| 37 | 35.4 | 2.65787 | 17.0 |
| 38 | 37.3 | 2.54264 | 13.0 |
| 39 | 38.1 | 2.49808 | 16.3 |
| 40 | 38.8 | 2.45667 | 16.3 |

### Comparative Example b Crystal Form F of the Compound of Formula (I)

The crystal form B of the present invention was placed in a methanol solvent, wherein the methanol solvent contained 5% water. The solution was heated to 50° C., and suspended at this temperature for 21 hours. And then the suspension was centrifuged to obtain the crystal form F of the compound of formula (I).

The XRPD pattern of the crystal form F was as shown in FIG. 20.

The XRPD pattern data of the crystal form F was shown in the following table:

TABLE 6
XRPD pattern analysis data of crystal form F

| | 2θ | Interplanar | Relative |
|---|---|---|---|
| Number | angle(°) | distance (Å) | intensity(%) |
| 1 | 4.4 | 19.88114 | 33.7 |
| 2 | 5.6 | 15.75133 | 13.0 |
| 3 | 7.3 | 12.07027 | 100.0 |
| 4 | 9.0 | 9.90494 | 27.1 |
| 5 | 10.3 | 8.58338 | 24.9 |
| 6 | 11.3 | 7.88158 | 43.9 |
| 7 | 11.5 | 7.75676 | 8.6 |
| 8 | 11.8 | 7.52708 | 9.3 |
| 9 | 12.2 | 7.31866 | 6.9 |
| 10 | 12.8 | 6.97041 | 8.1 |
| 11 | 13.5 | 6.61631 | 14.8 |
| 12 | 13.8 | 6.47388 | 6.6 |
| 13 | 14.1 | 6.32606 | 29.4 |
| 14 | 14.9 | 6.00854 | 21.0 |
| 15 | 15.3 | 5.85197 | 6.9 |
| 16 | 15.7 | 5.69433 | 10.4 |
| 17 | 16.0 | 5.57176 | 8.6 |
| 18 | 16.7 | 5.35461 | 6.6 |
| 19 | 17.0 | 5.27817 | 10.4 |
| 20 | 17.3 | 5.16957 | 5.7 |
| 21 | 18.0 | 4.98336 | 29.4 |
| 22 | 18.2 | 4.93813 | 23.0 |
| 23 | 19.1 | 4.69938 | 4.6 |
| 24 | 19.4 | 4.62913 | 12.5 |
| 25 | 20.2 | 4.46986 | 5.3 |
| 26 | 21.2 | 4.26553 | 6.7 |
| 27 | 21.8 | 4.14798 | 7.4 |
| 28 | 22.1 | 4.09409 | 8.5 |
| 29 | 22.3 | 4.06430 | 8.8 |
| 30 | 22.5 | 4.02604 | 9.8 |
| 31 | 22.7 | 3.99950 | 10.6 |
| 32 | 23.1 | 3.92422 | 5.3 |
| 33 | 23.6 | 3.83987 | 5.8 |
| 34 | 24.1 | 3.77480 | 8.3 |
| 35 | 24.4 | 3.73653 | 6.9 |
| 36 | 24.9 | 3.65348 | 6.4 |
| 37 | 25.7 | 3.54720 | 3.5 |
| 38 | 26.4 | 3.45870 | 3.5 |
| 39 | 27.6 | 3.32025 | 4.0 |
| 40 | 28.2 | 3.25632 | 4.8 |
| 41 | 29.2 | 3.16226 | 4.4 |

### Comparative Example c Crystal Form G of the Compound of Formula (I)

The preparation procedures of the crystal form G was similar to that of Example 2, except that the solvent used was tetrahydrofuran, and the feeding molar ratio of the compound of formula (I) to citric acid was 1:1.

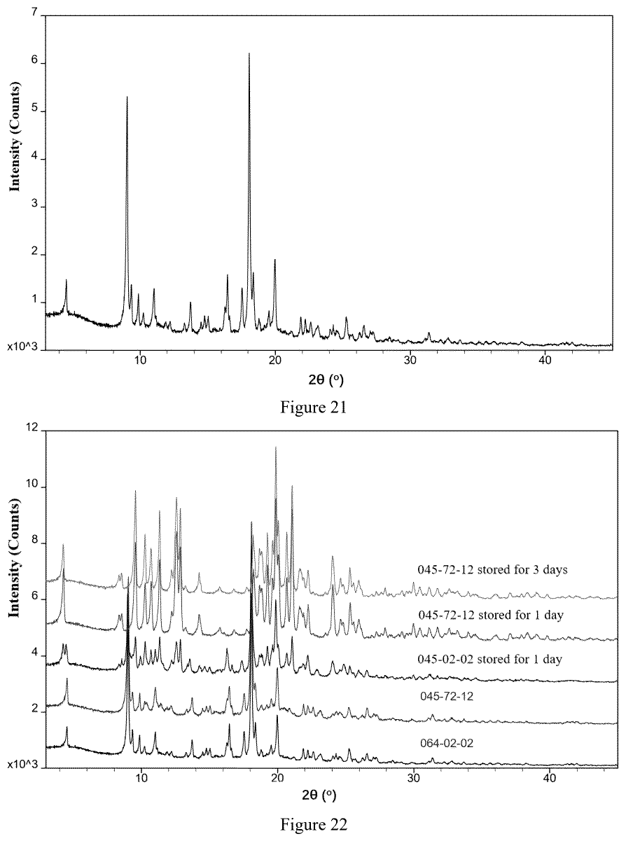

The XRPD pattern of the crystal form G was as shown in FIG. 21.

The XRPD pattern data of the crystal form G was shown in the following table:

TABLE 7
XRPD pattern analysis data of crystal form G

| | 2θ | Interplanar | Relative |
|---|---|---|---|
| Number | angle(°) | distance (Å) | intensity(%) |
| 1 | 4.5 | 19.47914 | 23.9 |
| 2 | 9.0 | 9.80506 | 85.4 |
| 3 | 9.4 | 9.47756 | 22.2 |
| 4 | 9.9 | 8.97837 | 19.1 |
| 5 | 10.2 | 8.67052 | 12.7 |
| 6 | 11.0 | 8.04821 | 20.9 |
| 7 | 11.9 | 7.48606 | 9.4 |
| 8 | 12.2 | 7.27223 | 9.8 |
| 9 | 13.3 | 6.70621 | 9.3 |
| 10 | 13.7 | 6.4921 | 16.3 |
| 11 | 14.5 | 6.14671 | 9.6 |
| 12 | 14.8 | 6.0451 | 11.7 |
| 13 | 15.0 | 5.93171 | 11.6 |
| 14 | 16.3 | 5.49736 | 14.7 |
| 15 | 16.5 | 5.43342 | 25.6 |
| 16 | 16.6 | 5.38337 | 11.7 |
| 17 | 17.6 | 5.1059 | 21.1 |
| 18 | 18.1 | 4.96238 | 100 |
| 19 | 18.4 | 4.88363 | 26.3 |
| 20 | 18.8 | 4.77835 | 10.9 |
| 21 | 19.5 | 4.60522 | 13.4 |
| 22 | 20.0 | 4.50644 | 30.7 |
| 23 | 21.9 | 4.13141 | 11.4 |
| 24 | 22.2 | 4.07113 | 10.6 |
| 25 | 22.6 | 4.00389 | 9.6 |
| 26 | 23.0 | 3.94328 | 6.8 |
| 27 | 23.2 | 3.91371 | 8.1 |
| 28 | 24.2 | 3.77867 | 7.2 |
| 29 | 24.3 | 3.74412 | 7.8 |
| 30 | 24.6 | 3.70653 | 6.6 |
| 31 | 24.6 | 3.69726 | 6.6 |
| 32 | 25.3 | 3.61083 | 11.1 |
| 33 | 25.7 | 3.55057 | 5.4 |
| 34 | 26.2 | 3.4844 | 5.6 |
| 35 | 26.6 | 3.44442 | 8.1 |
| 36 | 27.0 | 3.38864 | 6.7 |
| 37 | 27.2 | 3.36449 | 6.2 |
| 38 | 31.4 | 2.9591 | 6 |

### Test Examples

### Test Example 1 Test of Solubility of Salts of the Compound of Formula (I)

About 4-20 mg of salt of the compound of formula (I) was weighed, and the salt was added to the EP tube. A certain amount of solvent was added successively at room temperature. The solution was stirred, and it was observed whether the solid was completely dissolved. If the solid had not been completely dissolved after 10.0 mL of solvent was added, the experiment was stopped. The solubility of the compound in the solvent was estimated based on the volume of solvent used when the solid was completely dissolved. The experimental results were shown in the following table.

TABLE 8
Test results of solubility of salts of the compound of formula (I)

| Salt forms of | Amount of | Amount of | | |
|---|---|---|---|---|
| the compound | added sample | added water | Solution | Solubility |
| of formula (I) | (mg) | (mL) | state | (mg/mL) |
| Hydrochloride salt | 5.0 | 0.1 | clear | >50 |
| Sulfate salt | 5.0 | 0.7 | clear | >7.1 |
| Hydrobromide salt | 5.1 | 0.1 | clear | >51 |
| Mesylate salt | 4.7 | 0.1 | clear | >47 |
| p-Tosylate | 4.9 | 0.8 | clear | >6.1 |
| Maleate salt | 5.2 | 0.6 | clear | >8.6 |
| Fumarate salt | 5.2 | 0.6 | clear | >8.6 |
| Oxalate salt | 4.9 | 1.6 | clear | >3.0 |
| L-Tartrate | 4.7 | 0.1 | clear | >47 |
| Succinate salt | 4.8 | 0.1 | clear | >48 |
| Citrate salt | 19.8 | 0.6 | clear | >33 |

It could be seen from the above solubility test results that, compared with other salt forms, the hydrochloride salt, hydrobromide salt, mesylate salt, L-tartrate salt, succinate salt and citrate salt of the compound of formula (I) had excellent solubility. In the present test example, the citrate salt of the compound of formula (I) included the monocitrate salt of the compound of formula (I) (the molar ratio of the compound of formula (I) to citric acid was 1:1) and the dicitrate salt of the compound of formula (I) (the molar ratio of the compound of formula (I) to citric acid is 1:2).

### Test Example 2 Hygroscopicity of Salts of the Compound of Formula (I)

The compound of formula (I) was placed in a humid environment, wherein the initial humidity of the environment was 50%, and the humidity was increased to 95% in increments of 15%. The maintenance time for each humidity gradient was 60 min. After the ambient humidity was maintained at 95% for 60 min, the weight gain of each salt form was tested to determine the hygroscopicity of the corresponding salt form.

TABLE 9
Test results of hygroscopicity of salts
of the compound of formula (I)

| Salt forms of the compound of formula (I) | Weight gain at 95% humidity |
|---|---|
| Hydrochloride salt | 10.97% |
| Sulfate salt | 7.04% |
| Hydrobromide salt | 14.02% |
| Mesylate salt | 12.00% |
| p-Tosylate | 3.44% |
| Maleate salt | 2.55% |
| Fumarate salt | 2.54% |
| Oxalate salt | 4.01% |
| L-Tartrate | 8.18% |
| Succinate salt | 26.28% |
| Citrate salt | 0.64% |

According to the above hygroscopicity test results, it could be seen that compared with other salt forms, p-tosylate salt, maleate salt, fumarate salt, oxalate salt and citrate salt of the compound of formula (I) had lower weight gain, confirming that these salt forms had improved hygroscopicity, and had more excellent stability in humid environment.

In addition, according to the results of Text Examples 1-2, it could be seen that, compared with other salt forms, the citrate salt of the compound of formula (I) had more excellent comprehensive properties, and its solubility and hygroscopicity were superior to other salt forms.

### Test Example 3 Solubility and Hygroscopicity Study of Citrate Salt of the Compound of Formula (I)

According to the test procedures of Test Example 1, the solubility of the monocitrate salt and dicitrate salt of the compound of formula (I) were respectively tested. The test results obtained were as shown in the following Table 10.

According to the test procedures of Test Example 2, the solubility of the monocitrate salt and dicitrate salt of the compound of formula (I) were respectively tested. The test results obtained were as shown in the following Table 11.

TABLE 10
The test results of solubility of the citrate
salt of the compound of formula (I)

| Salt forms of | Amount of | Amount of | | |
|---|---|---|---|---|
| the compound | added sample | added water | Solution | Solubility |
| of formula (I) | (mg) | (mL) | state | (mg/mL) |
| Monocitrate salt | 19.7 | 0.6 | clear | >33 |
| Dicitrate salt | 19.8 | 0.6 | clear | >33 |

TABLE 11
The test results of hygroscopicity of the
citrate salt of the compound of formula (I)

| Salt forms of the compound of formula (I) | Weight gain at 95% humidity |
|---|---|
| Monocitrate salt | 3.45% |
| Dicitrate salt | 0.64% |

According to the test results in Table 10 and Table 11, it could be seen that the dicitrate salt of the compound of formula (I) (the molar ratio of the compound of formula (I) to citric acid was 1:2) had relatively more excellent comprehensive properties. The above test results confirmed that in the citrate salt of the compound of (I), an appropriate molar ratio of the compound of formula (I) to citric acid was beneficial for the salt forms to have more excellent solubility and hygroscopicity.

### Test Example 4 the Moisture Absorption Study of Crystal Forms of the Compound of Formula (I)

Dynamic vapor sorption and desorption analysis of crystal forms were performed using DVS Intrinsic (SMS, UK) to evaluate the hygroscopicity of the crystal forms. The gradient mode was used for the test. The humidity change was 50%-95%-50%. The humidity change amount of each gradient was 15%. The gradient endpoint was determined in the dm/dt manner, with dm/dt less than 0.002% and maintained for 10 minutes as the gradient endpoint, or the maximum maintenance time of each gradient was 60 min. After the test was completed, XRPD analysis was performed on the sample to confirm whether the solid form changed.

### Crystal Form A

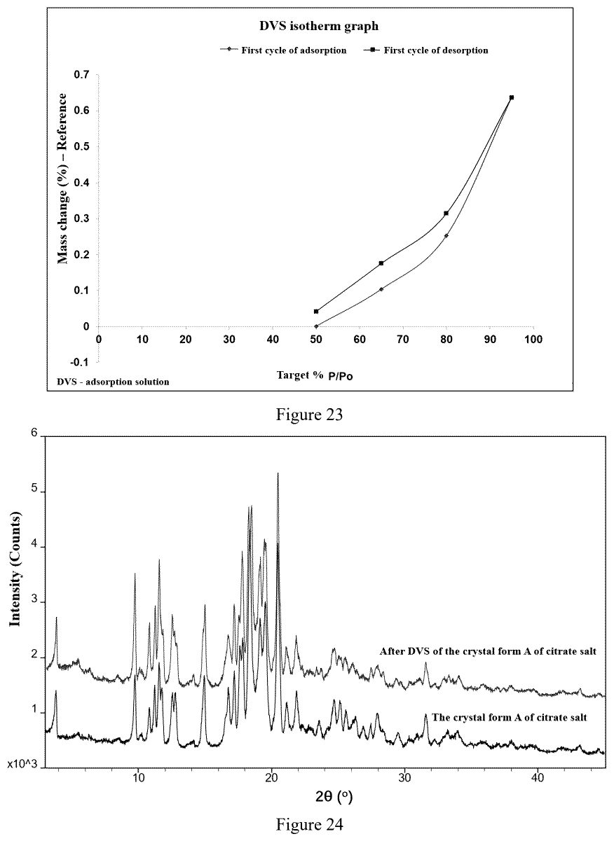

As shown in FIG. 23, with reference to the mass under the condition of 50% RH, the weight of the crystal form A increased by 0.64% at 95% RH, and during the adsorption process, the weight increased by 0.25% at 80% RH.

As shown in FIG. 24, the XRPD pattern of the crystal form A after testing did not change significantly.

The above results showed that after experiencing a cycle of humidity changes, the weight gain of the crystal form A was lower, and the crystal form A had not undergone crystal form transformation, confirming that the crystal form A had excellent humidity stability.

### Crystal Form B

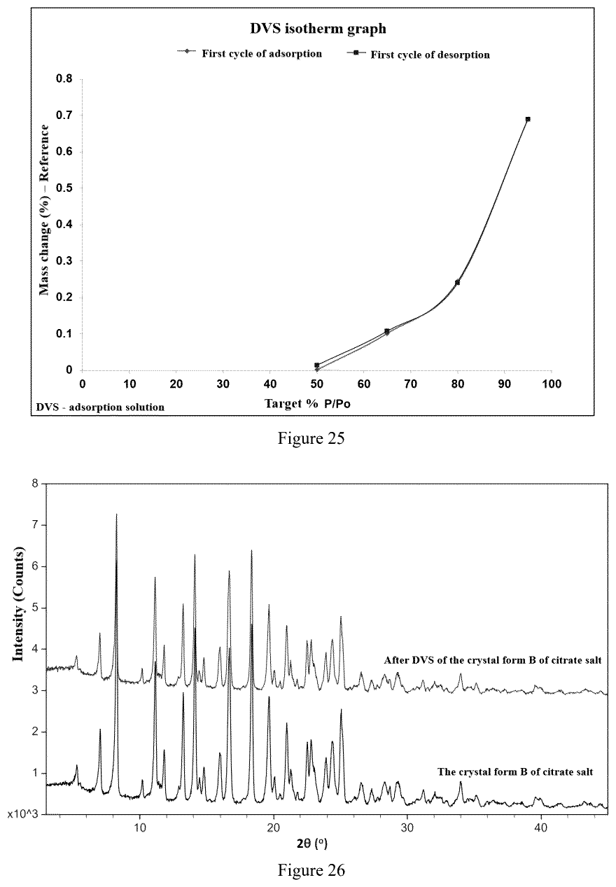

As shown in FIG. 25, with reference to the mass under the condition of 50% RH, the weight of the crystal form B increased by 0.69% at 95% RH, and during the adsorption process, the weight increased by 0.24% at 80% RH.

As shown in FIG. 26, the XRPD pattern of the crystal form B after testing did not change significantly.

The above results showed that after experiencing a cycle of humidity changes, the weight gain of the crystal form B was lower, and the crystal form B had not undergone crystal form transformation, confirming that the crystal form B had excellent humidity stability.

### Crystal Form C

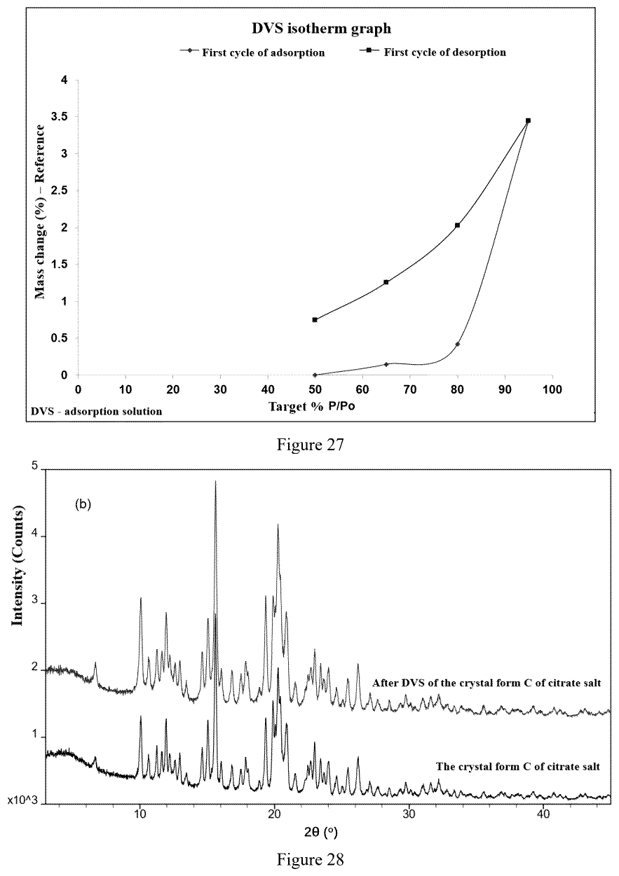

As shown in FIG. 27, with reference to the mass under the condition of 50% RH, the weight of crystal form C increased by 3.45% at 95% RH, and during the adsorption process, the weight increased by 0.42% at 80% RH.

As shown in FIG. 28, the XRPD pattern of the crystal form C after testing did not change significantly.

The above results showed that after experiencing a cycle of humidity changes, the weight gain of the crystal form C was lower, and the crystal form C had not undergone crystal form transformation, confirming that the crystal form C had excellent humidity stability.

### Crystal Form D

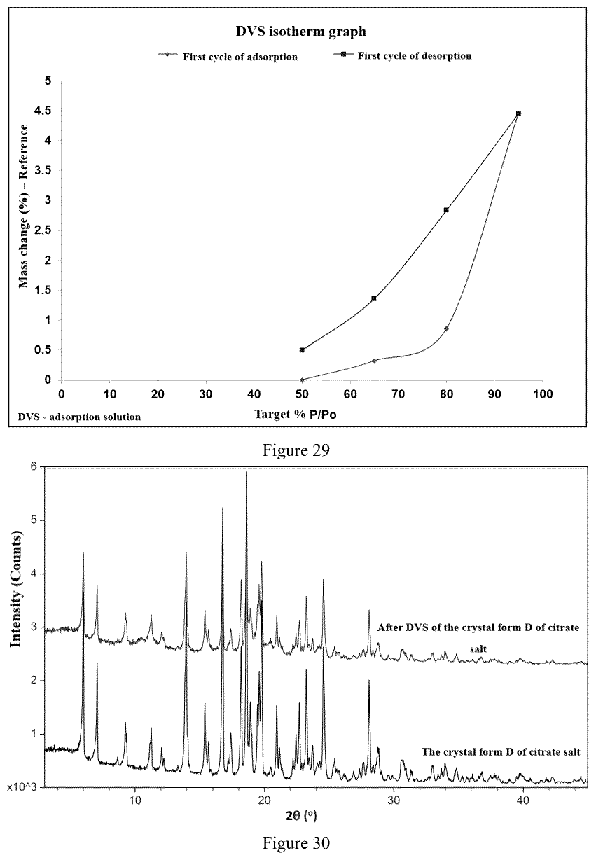

As shown in FIG. 29, with reference to the mass under the condition of 50% RH, the weight of the crystal form D increased by 4.45% at 95% RH, and during the adsorption process, the weight only increased by 0.85% at 80% RH.

As shown in FIG. 30, the XRPD pattern of the crystal form D after testing did not change significantly.

The above results showed that after experiencing a cycle of humidity changes, the weight gain of the crystal form D was lower, and the crystal form D had not undergone crystal form transformation significantly, confirming that the crystal form D had better humidity stability.

### Test Example 5 the Stability Study of Crystal Forms of the Compound of Formula (I)

Stability studies under conditions of high temperature (60° C.), high humidity (25° C./92.5% RH), illumination (25° C./4500 Lux), and accelerated condition (40° C./75% RH) were performed on the crystal forms of the compound of formula (I). Samples were taken for XRPD characterization on the 7th and 15th days respectively.

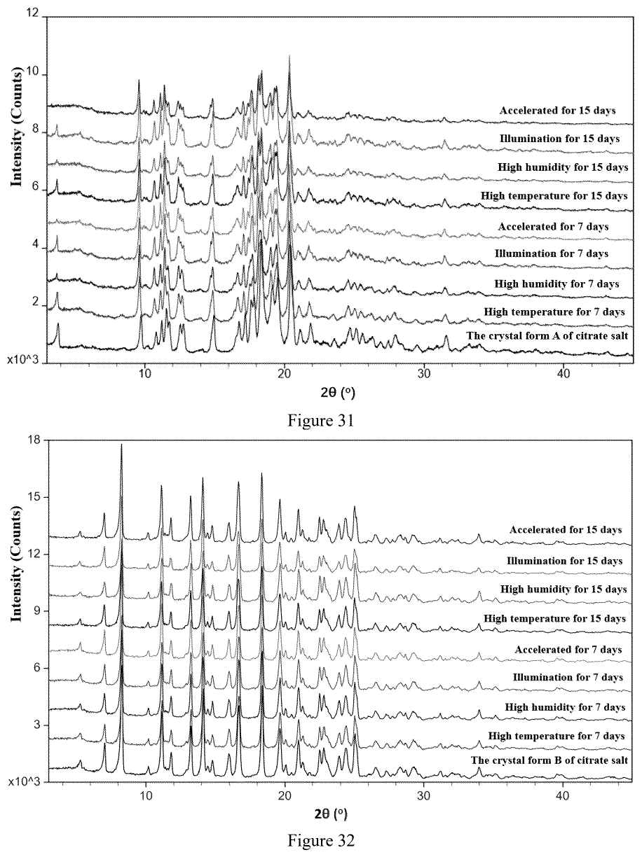

The above stability studies were performed on the crystal form A, and the test results were as shown in FIG. 31. The results of XRPD showed that the crystal form A was stable under high temperature, high humidity, illumination and accelerated conditions for 15 days, without any crystal form transformation occurring.

The above test results confirmed that the crystal form A of the compound of formula (I) had excellent stability.

The above stability studies were performed on the crystal form B, and the test results were as shown in FIG. 32. The results of XRPD showed that the crystal form B did not undergo crystal form transformation after 7 days and 15 days under high temperature, high humidity, illumination and accelerated conditions.

The above test results confirmed that the crystal form B of the compound of formula (I) had excellent stability.

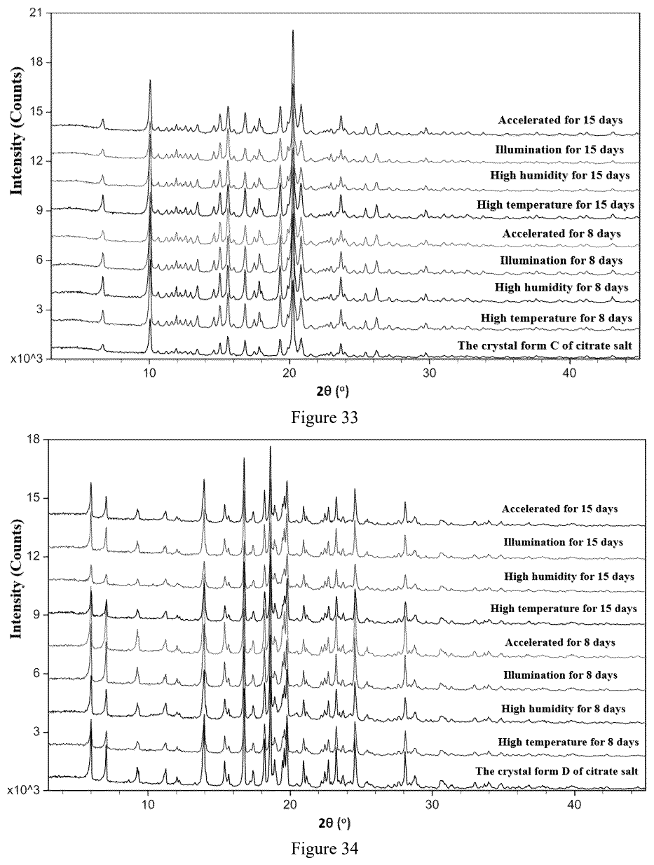

The above stability studies were performed on the crystal form C, and the test results were as shown in FIG. 33. The results of XRPD showed that the crystal form C did not undergo crystal form transformation after 8 days and 15 days under high temperature, high humidity, illumination and accelerated conditions.

The above test results confirmed that the crystal form C of the compound of formula (I) had excellent stability.

The above stability studies were performed on the crystal form D, and the test results were as shown in FIG. 34. The XRPD results showed that the crystal form D did not undergo significant crystal form transformation after being maintained under high humidity, high temperature, illumination, and accelerated conditions for 8 and 15 days.

The above test results confirmed that the crystal form D of the compound of formula (I) had excellent stability.

The crystal form G was stored at room temperature for 1 day. The XRPD pattern showed (refer to FIG. 22) that the crystal form G underwent crystal form transformation after being stored at room temperature for 1 day.

The above results showed that the crystal forms A, B, C and D had improved stability compared with other crystal forms of the compound of formula (I). In particular, the crystal forms A, B, and C had significantly improved stability compared with other crystal forms of the compound of formula (I).

### Test Example 6 Biological Media and Water Solubility Test of Salt Forms and Crystal Forms of the Compound of Formula (I)

The crystal form A of the citrate salt of the compound of formula (I) was added to water or three biological media (FaSSIF, FeSSIF and FaSSGF) to determine the solubility in the above solvents. Wherein, FaSSIF could be used to simulate the intestinal fluid in the small intestine in the human pre-prandial hunger state; FeSSIF could be used to simulate the intestinal fluid in the small intestine in the human post-prandial satiety state; FaSSGF could be used to simulate the stomach in the empty stomach in the human hunger state.

The results showed that the crystal form A was suspended in four media, dissolved in 0.5 h, and the solution was still clear after suspension for 24 h. The solubility of the citrate salt of the compound of formula (I) in the four media decreased in the order of FaSSGF>FeSSIF>FaSSIF>water. The pH of supernatant of the crystal form A decreased to different degrees after FaSSIF and FeSSIF solubility was tested, and the pH of supernatant increased after FaSSGF solubility was tested.

The above results confirmed that the crystal form A of the compound of formula (I) had excellent solubility in FaSSIF and water. It could be seen that the crystal form A of the compound of formula (I) of the present invention had excellent solubility.

TABLE 12
Dynamic solubility test in biological media and water

| | | | Amount of | Solubility (mg/mL) | | | Final | XRPD of the |
|---|---|---|---|---|---|---|---|---|
| Solid form | Medium | pH | sample (mg) | 0.5 h | 2 h | 24 h | pH | remaining solid |
| Crystal form | FaSSIF | 6.5 | 39.9 | >5.15 | >5.11 | >5.18 | 4.3 | No solid remained |
| A of the | FeSSIF | 5.0 | 39.6 | >5.46 | >5.48 | >5.47 | 4.6 | No solid remained |
| compound of | FaSSGF | 1.6 | 40.2 | >5.47 | >5.48 | >5.38 | 2.5 | No solid remained |
| formula (I) | Water | | 40.3 | >5.54 | >5.45 | >5.48 | 3.8 | No solid remained |

### Test Example 7 Transformation of Crystal Forms of Compound of Formula (I)

It could be seen from the preparation of the above comparative examples that:

(1) when the crystal form B of the compound of formula (I) was heated in situ to 81° C., the crystal form E of the compound of formula (I) could be obtained;

However, after the heating was stopped and the temperature dropped to room temperature, the crystal form E would transform back into the crystal form B;

(2) the crystal form B was placed in a methanol solvent containing 5% water. The solution was heated to 50° C. and suspended at this temperature for 21 hours, and then the crystal form F could be obtained;

After the above suspension was stored under normal temperature and humidity, the crystal form F would transform back into the crystal form B;

It could be seen that the crystal form E or F could be transformed into the crystal form B under normal temperature conditions. That is to say, it was confirmed that compared with the crystal form E or F, the crystal form B had more excellent stability.

The above descriptions are merely specific embodiments of the present invention, and are not intended to limit the patent scope of the present invention. Any equivalent transformation made by using the present invention, or direct or indirect application in other related technical fields is similarly included in the patent protection scope of the present invention.

1. A citrate salt of a compound of formula (I)

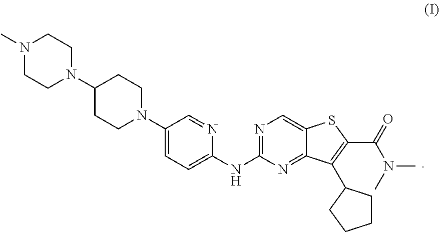

2. The citrate salt of the compound of formula (I) according to claim 1, wherein
the molar ratio of the compound of formula (I) to citric acid is about 1:1-1:3, preferably about 1:1-1:2, more preferably about 1:2.

3. A crystal form of a citrate salt of a compound of formula (I) 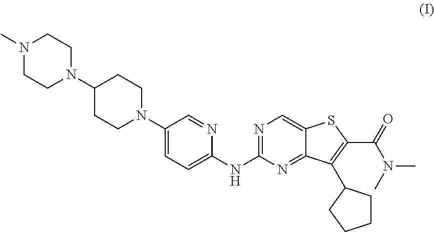
which is selected from the group consisting of
(1) A crystal form A of the citrate salt of the compound of formula (I), wherein
the X-ray powder diffraction pattern of the crystal form A has characteristic diffraction peaks at the following 2θ angles: 17.9±0.2°, 18.4±0.2°, 19.2±0.2°, 19.5±0.2° and 20.5±0.2°;
preferably, the X-ray powder diffraction pattern of the crystal form A has characteristic diffraction peaks at the following 2θ angles: 9.8±0.2°, 11.6±0.2°, 15.0±0.2°, 17.2±0.2°, 17.6±0.2°, 17.9±0.2°, 18.4±0.2°, 19.2±0.2°, 19.5±0.2° and 20.5±0.2°;
more preferably, the X-ray powder diffraction pattern of the crystal form A has characteristic diffraction peaks at the following 2θ angles: 3.8±0.2°, 9.8±0.2°, 11.2±0.2°, 11.6±0.2°, 11.8±0.2°, 12.6±0.2°, 12.8±0.2°, 15.0±0.2°, 16.8±0.2°, 17.2±0.2°, 17.6±0.2°, 17.9±0.2°, 18.4±0.2°, 19.2±0.2°, 19.5±0.2°, 20.5±0.2°, 21.1±0.2°, 21.9±0.2°, 24.7±0.2°, and 25.2±0.2°;
particularly preferably, the X-ray powder diffraction pattern of the crystal form A is as shown in FIG. 1;
(2) crystal form B of the citrate salt of the compound of formula (I), wherein
the X-ray powder diffraction pattern of the crystal form B has characteristic diffraction peaks at the following 2θ angles: 8.3±0.2°, 11.2±0.2°, 14.1±0.2°, 16.7±0.2° and 18.4±0.2°;
preferably, the X-ray powder diffraction pattern of the crystal form B has characteristic diffraction peaks at the following 2θ angles: 5.3±0.2°, 7.0±0.2°, 8.3±0.2°, 11.2±0.2°, 11.8±0.2°, 14.1±0.2°, 14.8±0.2°, 16.0±0.2°, 16.7±0.2°, 18.4±0.2°, 22.5±0.2°, 22.8±0.2°, 23.9±0.2°, 24.4±0.2°, and 25.0±0.2°;
more preferably, the X-ray powder diffraction pattern of the crystal form B has characteristic diffraction peaks at the following 2θ angles: 5.3=0.2°, 7.0±0.2°, 8.3±0.2°, 10.2±0.2°, 11.2±0.2°, 11.8±0.2°, 13.2±0.2°, 14.1±0.2°, 14.8±0.2°, 16.0±0.2°, 16.7±0.2°, 18.4±0.2°, 19.7±0.2°, 20.1±0.2°, 21.0±0.2°, 21.3±0.2°, 22.5±0.2°, 22.8±0.2°, 23.0±0.2°, 23.9±0.2°, 24.4±0.2°, and 25.0±0.2°;
particularly preferably, the X-ray powder diffraction pattern of the crystal form B is as shown in FIG. 6;
(3) crystal form C of the citrate salt of the compound of formula (I), wherein
the X-ray powder diffraction pattern of the crystal form C has characteristic diffraction peaks at the following 2θ angles: 10.0±0.2°, 15.6±0.2°, 19.4±0.2°, 20.2±0.2° and 20.8±0.2°;
preferably, the X-ray powder diffraction pattern of the crystal form C has characteristic diffraction peaks at the following 2θ angles: 6.7±0.2°, 10.0±0.2°, 11.9±0.2°, 15.0±0.2°, 15.6±0.2°, 16.8±0.2°, 19.4±0.2°, 19.9±0.2°, 20.2±0.2° and 20.8±0.2°;
more preferably, the X-ray powder diffraction pattern of the crystal form C has characteristic diffraction peaks at the following 2θ angles: 6.7±0.2°, 10.0±0.2°, 10.6±0.2°, 11.2±0.2°, 11.9±0.2°, 12.6±0.2°, 13.0±0.2°, 13.4±0.2°, 14.6±0.2°, 15.0±0.2°, 15.6±0.2°, 16.0±0.2°, 16.8±0.2°, 17.5±0.2°, 17.8±0.2°, 18.0±0.2°, 19.4±0.2°, 19.9±0.2°, 20.2±0.2°, 20.8±0.2°, 23.0±0.2°, 23.7±0.2°, 25.4±0.2°, and 26.2±0.2°;
particularly preferably, the X-ray powder diffraction pattern of the crystal form C is as shown in FIG. 10;
(4) crystal form D of the citrate salt of the compound of formula (I), wherein
the X-ray powder diffraction pattern of the crystal form D has characteristic diffraction peaks at the following 2θ an 14.0±0.2°, 16.8±0.2°, 18.6±0.2°, 19.8±0.2°, 24.6±0.2°;
preferably, the X-ray powder diffraction pattern of the crystal form D has characteristic diffraction peaks at the following 2θ angles: 6.0±0.2°, 7.1±0.2°, 14.0±0.2°, 15.4±0.2°, 16.8±0.2°, 18.2±0.2°, 18.6±0.2°, 19.6±0.2°, 19.8±0.2°, 23.3±0.2°, 24.6±0.2° and 28.1±0.2°;
more preferably, the X-ray powder diffraction pattern of the crystal form D has characteristic diffraction peaks at the following 2θ angles: 6.0±0.2°, 7.1±0.2°, 9.2±0.2°, 11.3±0.2°, 12.1±0.2°, 14.0±0.2°, 15.4±0.2°, 15.7±0.2°, 16.8±0.2°, 17.4±0.2°, 18.2±0.2°, 18.6±0.2°, 19.0±0.2°, 19.6±0.2°, 19.8±0.2°, 21.0±0.2°, 21.2±0.2°, 22.4±0.2°, 22.7±0.2°, 23.3±0.2°, 23.5±0.2°, 23.7±0.2°, 24.6±0.2°, 28.1±0.2° and 28.8±0.2°;
particularly preferably, the X-ray powder diffraction pattern of the crystal form D is as shown in FIG. 15.

4. The crystal form of the citrate salt of the compound of formula (I) according to claim 3, wherein
the differential scanning calorimetry analysis curve of the crystal form A has endothermic peaks at 190.1±3° C.; preferably, the differential scanning calorimetry analysis curve of the crystal form A is as shown in FIG. 2;
(2) the differential scanning calorimetry analysis curve of the crystal form B has endothermic peaks at 81.0±3° C., 133.2±3° C., 150.6±3° C. and 177.9±3° C., respectively; preferably, the differential scanning calorimetry analysis curve of the crystal form B is shown in FIG. 7;
(3) the differential scanning calorimetry curve of the crystal form C has endothermic peaks at 194.3±3° C., preferably, the differential scanning calorimetry curve of the crystal form C is as shown in FIG. 11; and/or
(4) the differential scanning calorimetry curve of the crystal form D has endothermic peaks at 148.4±3° C., 160.8° C. and 184.1° C., respectively, preferably, the differential scanning calorimetry curve of the crystal form D is as shown in FIG. 16.

5. (canceled)

6. (canceled)

7. (canceled)

8. (canceled)

9. (canceled)

10. (canceled)

11. A process for preparing the crystal form of the citrate salt of the compound of formula (I) according to claim 3, wherein the crystal form is the crystal form A of the citrate salt of the compound of formula (I), comprising the following steps:
providing a solution of citric acid;
providing a solution of the compound of formula (I) in a solvent;
adding the solution of citric acid to the solution of the compound of formula (I);
stirring the obtained solution and cooling;
filtering the cooled solution and drying the filtrate to obtain the crystal form A of the compound of formula (I);
wherein the solvent is selected from the group consisting of an alcohol solvent, an ether solvent, a nitrile solvent, a ketone solvent, and a combination thereof;
preferably,
the alcohol solvent is selected from the group consisting of methanol, ethanol, isopropanol, n-propanol, n-butanol, and a combination thereof, more preferably is selected from the group consisting of methanol, ethanol, isopropanol, and a combination thereof;
the ether solvent is selected from the group consisting of ethyl ether, ethylene glycol methyl ether, ethylene glycol dimethyl ether and a combination thereof, more preferably is selected from the group consisting of ethylene glycol methyl ether, ethylene glycol dimethyl ether and a combination thereof;
the nitrile solvent is selected from the group consisting of acetonitrile, propionitrile, and a combination thereof, more preferably is acetonitrile;
the ketone solvent is selected from the group consisting of acetone, butanone, 4-methyl-2-pentanone, and a combination thereof, more preferably is selected from the group consisting of acetone, 4-methyl-2-pentanone, and a combination thereof.

12. The process according to claim 11, wherein the molar ratio of the compound of formula (I) to the citric acid is about 1:3-1:5, preferably about 1:3-1:4.

13. A process for preparing the crystal form of the citrate salt of the compound of formula (I) according to claim 3, wherein the crystal form is the crystal form B of the citrate salt of the compound of formula (I), comprising the following steps:
providing a solution of citric acid;
providing a solution of the compound of formula (I) in a solvent;
adding the solution of citric acid to the solution of the compound of formula (I);
stirring the obtained solution and cooling;
filtering the cooled solution and drying the filtrate to obtain the crystal form B of the compound of formula (I);
wherein the solvent of is selected from the group consisting of an alcohol solvent, water, and a combination thereof;
preferably,
the alcohol solvent is selected from the group consisting of methanol, ethanol, isopropanol, n-propanol, and a combination thereof, preferably is selected from the group consisting of methanol, ethanol, and a combination thereof.

14. The process according to claim 13, wherein the molar ratio of the compound of formula (I) to the citric acid is about 1:3-1:5, preferably about 1:3-1:4.

15. The process according to claim 13, wherein the mass ratio of the alcohol solvent to water is about 10:1-3:1, preferably about 6:1-4:1.

16. A process for preparing the crystal form of the citrate salt of the compound of formula (I) according to claim 3, wherein the crystal form is the crystal form C of the citrate salt of the compound of formula (I), comprising the following steps:
adding a citrate salt of the compound of formula (I) to solvent X to obtain a solution;
adding solvent Y to the above solution and stirring at room temperature;
obtaining the crystal form C by centrifugation;
wherein
the solvent X is selected from the group consisting of dimethylformamide, ethylene glycol methyl ether, trifluoroethanol, and a combination thereof;
the solvent Y is selected from the group consisting of acetonitrile, ethyl acetate, methanol, ethanol, isopropanol, and a combination thereof.

17. A pharmaceutical composition comprising
the citrate salt of the compound of formula (I) according to claim 1.

18. (canceled)

19. (canceled)

20. (canceled)

21. A method for treating, ameliorating, or preventing an abnormal cell proliferation comprising administering to a subject in need thereof an effective amount of the citrate salt of the compound of formula (I) according to claim 1 or a pharmaceutical composition thereof, and optionally in combination with a second therapeutic agent.

22. The crystal form of the citrate salt of the compound of formula (I) according to claim 3, wherein
(1) the thermogravimetric analysis curve of the crystal form A has a weight loss of 1.2% at 120±3° C. and a weight loss of 38.4% at 140-300±3° C.; preferably, the thermogravimetric analysis curve of the crystal form A is as shown in FIG. 3;
(2) the thermogravimetric analysis curve of the crystal form B has a weight loss of 2.2% at 90±3° C., a weight loss of 1.8% at 90-140±3° C., and a weight loss of 36.9% at 140-300±3° C.; preferably, thermogravimetric analysis curve of the crystal form B is as shown in FIG. 8;
(3) the thermogravimetric analysis curve of the crystal form C has a weight loss of 0.1% at 120±3° C. and a weight loss of 25.8% at 120-300±3° C.; preferably, the thermogravimetric analysis curve of the crystal form C is as shown in FIG. 12; and/or
(4) the thermogravimetric analysis curve of the crystal form D has a weight loss of 0.6% at 110±3° C., a weight loss of 1.7% at 110-150±3° C., and a weight loss of 27.7% at 150-300±3° C.; preferably, thermogravimetric analysis curve of the crystal form D is as shown in FIG. 17.

23. A pharmaceutical composition comprising one or more the crystal form of the citrate salt of the compound formula (I) according to claim 3.

24. A method for treating, ameliorating, or preventing an abnormal cell proliferation comprising administering to a subject in need of such treatment an effective amount of the crystal form of the citrate salt of the compound of formula (I) according to claim 3 or a pharmaceutical composition thereof, and optionally in combination with a second therapeutic agent.
